(19) United States
(12) Patent Application Publication
(10) Pub. No.: US 2004/0075811 A1
(43) Pub. Date: Apr. 22, 2004

(54) Device and method for exercising eyes

(75) Inventor: Jacob Liberman, Kula, HI (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Exercise Your Eyes, Inc., New York, NY

(21) Appl. No.: 10/345,757

(22) Filed: Jan. 16, 2003

Related U.S. Application Data
(63) Continuation-in-part of application No. 10/123,594, filed on Apr. 16, 2002.

(51) Int. Cl. A61B 3/00

(52) U.S. Cl. 351/203

(57) ABSTRACT

A system and medium is provided for exercising an observer's eyes. It includes a display device, a computer processor with associated computer memory, the processor operatively coupled to the display, the computer memory, and one or more input devices, the computer processor being configured to display on the display device a predetermined sequence of colored images, the color of each colored image having varying wavelength, each successive colored image having a sufficiently different wavelength from its predecessor color image to cause the focusing power of the observer's eyes to adjust, each colored image being singly displayed for a period of time sufficient for the focusing power of an observer's eyes to adjust.

### CROSS-REFERENCE TO RELATED APPLICATIONS

This application is a continuation-in-part of non-provisional U.S. patent application Ser. No. 10/123,594, entitled “Device and Method for Exercising Eyes.”

### FIELD OF THE INVENTION

The present invention relates to devices and methods for exercising eyes.

### BACKGROUND OF THE INVENTION

Vision is the primary navigational system of a human body, providing 80 to 90% of all information received during a person's lifetime. The proficiency of the vision skills affects every human activity and affects human performance on all levels. However, the human vision system functions in a more and more difficult environment as educational and occupational demands continue to grow exponentially in today's society.

The United States economy, as well as that of many foreign countries, has moved from an industrial era to a service era and has entered the information age. More and more, a worker's performance depends on gathering and internalizing a growing body of information in educational, occupational, and even social surroundings.

The computer has become a principal channel for providing services and information. There is an ongoing and dramatic rise in the number of people who use computers at work, at home after work hours, while shopping, reading the newspaper, and the like. The volume of services and information provided via computers also continues to increase. The explosive growth in the use of computers and other vision-related information-gathering activities dramatically increases demands on the vision system.

The visual system and its primary instrument, the eyes, do not respond well to this increased demand. The eyes are meant to respond effortlessly to images of objects that enter awareness and call for attention. However, it is unlikely that the eyes were designed to be used primarily for reading or working on a computer. Yet, as already discussed above, the educational and occupational requirements lead people to do just that.

As a consequence, modern society suffers from a virtual epidemic of vision problems, especially myopia. Such vision problems, including myopia, can be directly related to the amount of time spent reading or working on a computer. The educational system, with its major focus on visual information transmission and communication, is a major contributor to the epidemic.

The eyes are complex neuro-optical systems of the human body. They locate, track, and focus on objects of interest. Before describing the structure and functioning of the eyes, it is useful to describe certain aspects of inanimate optics and related physical phenomena.

A human eye perceives electromagnetic radiation in a certain narrow range of wavelengths (˜400 nm to ˜700 nm), which may be referred to as the visible range. For the most part, the light perceived by the eye as images of various objects includes mixtures of light waves with different wavelengths. Thus, white light is a mixture of light waves of essentially all wavelengths in the visible range. The electromagnetic waves with unique wavelengths within the visible range (monochromatic light) are perceived as colors. For example, the monochromatic light with the wavelength of 660 nm is perceived as red and the light with the wavelength of 470 nm as blue. Various combinations of light waves (e.g., additions or subtractions) may also be perceived as colors.

On the basis of human perception of colors, the visible range is often divided into various color sub-ranges. One commonly described classification divides the visible range into violet, indigo, blue, green, yellow, orange, and red color sub-ranges:

| Color sub-range | Wavelengths (nm) |
|---|---|
| Violet | ˜400-425 |
| Indigo | ˜425-450 |
| Blue | ˜450-490 |
| Green | ˜490-570 |
| Yellow | ˜570-590 |
| Orange | ˜590-620 |
| Red | >˜620 |

Another classification divides the visible range into blue (<˜490 nm), green-yellow (˜490-590 nm), and red (>˜590 nm) sub-ranges. It should be noted that the boundaries between the color sub-ranges are approximate and depend on many factors. For additional discussion of human perception of color, see J. Liberman, Light: Medicine of the Future, Bear & Co., 1991.

Light interacts with material substances. Thus, light may, change direction when passing through material substances, a phenomenon known as refraction. An index of refraction (n) measures the magnitude of refraction for a given substance. The index of refraction of a substance is the ratio of the velocity of light in a vacuum (C) to the velocity (υ_{ν}) of the light wave with a particular wavelength (ν) in the substance: n=C/υ_{ν}. The velocity of light in a vacuum is constant. However, in material substances, the velocity of light is different for each wavelength ν. Therefore, the index of refraction is different at different wavelengths. For this reason, light waves of different wavelengths (colors) are refracted by different amounts through the same optical element. The index of refraction increases as wavelength decreases, and therefore colors of shorter wavelengths exhibit greater change in direction in material substances than colors of longer wavelengths.

The refraction of light is used in various optical systems, such as prisms, lenses, and the like, to manipulate light in a desired manner. A lens is an optical system bounded by two refracting surfaces having a common axis. Lenses refract and focus light emitted by or reflected from various objects. Each lens has a characteristic focus point and focal length, which are commonly used to describe lenses (FIG. 1). The focus point is a point at which the lens focuses light from an object located at an infinite distance from the lens.

Referring to FIG. 1, F₁ is the focus point of the lens L₁, and F₂ is the focus point of the lens L₂. The focal length or focal distance (f) is the distance from the center of the lens to its focus point. In the examples of FIG. 1, f₁ is the focal length of the lens L₁, and f₂ is the focal length of the lens L₂. The focal length f determines the properties of a lens with respect to focusing of light.

FIG. 2 illustrates how lenses focus light from an object. As seen in FIG. 2, the lens L captures light from an object located at a point Q. The light is focused into an image of the captured object at a point Q′. The point Q is known as the object point and the point Q′ as the image point. S denotes the distance from the object point Q to the lens L, and S′ denotes the distance from the lens L to the image point Q′.

For an ideal lens, one expression of the relationship between the focal length f and the distances S and S′ is the thin lens equation: 1/S+1/S′=1/f. If the object point Q is located at an infinite distance from the lens L (i.e., S is infinity), the term 1/s approaches zero and the image distance S′ is equal to the focal length of the lens L. If the object distance S is less than infinity, the distance S′ varies as a function of the distance S. Generally, for a given wavelength, the focal length f is fixed for a given inanimate lens. The term 1/f is also fixed for a given lens. Thus, the term 1/f is a parameter of the functional variation between the terms 1/S and 1/S′ (and therefore the distances S and S′). The term 1/f is known as the focusing power of the lens. The focusing power is measured in diopters, which is a metric unit equal to 1 divided by the focal length of the lens, in meters (1 diopter=1 m⁻¹). The shorter the focal length f of the lens, the greater the focusing power 1/f.

If the thin lens equation is applied to two different lenses with different focusing powers, the images of objects located at the same distance S are expected to be formed at different image distances S′. Referring again to FIG. 1, the focal length f₂ of the lens L₂ is greater than the focal length f₁ of the lens L₁, and thus the lens L₂ has more focusing power than the lens L₁. As seen from FIG. 1, the greater the focusing power of the lens, the closer to the lens the captured image is formed.

As explained above, the index of refraction (n) varies with the wavelength, and therefore, for the same lens, the magnitude of refraction is different for light of different wavelengths (colors). Thus, the focal length of the same lens is different for different colors. As a consequence, a single lens forms not one image of an object, but a series of images at varying distances from the lens, one for each color present in the light emitted or reflected by the object. If the lens captures monochromatic light, an observer placed at the focus point of the lens perceives the image as sharp. However, if the captured light is not monochromatic, some of the constituent light waves may remain unfocused. This phenomenon, known as chromatic aberration, is illustrated in FIG. 3.

Referring to FIG. 3, the lens L captures non-monochromatic light from an object AB. Suppose, the light from the object AB includes light waves having wavelengths ν₁ and ν₂ (light waves ν₁ and ν₂), where ν₁ <ν₂. Since the index of refraction is greater for shorter wavelengths, the lens L changes the direction of the light wave ν₁ more than the direction of the light wave ν₂. Therefore, the focal length of the lens L is smaller for the light wave ν₁ than for the wavelength ν₂.

The image for the light wave ν₁, shown as A′B′, is formed closer to the lens L than the image for the light wave ν₂, shown as A″B″. For example, if the wavelength ν₁ is in the violet color sub-range and the wavelength ν₂ is in the green color sub-range, the violet image would be formed closer to the lens L than the green image. The variation in the image distance as a function of color is called longitudinal chromatic aberration. The difference in the index of refraction at different wavelengths also affects the size of the image. The variation in the image size as a function of color is known as lateral chromatic aberration. In FIG. 3, the distance a measures the longitudinal chromatic aberration, and the distance b measures the lateral chromatic aberration.

Because of chromatic aberration, the same focus point is not optimal for all colors that comprise the light captured through the lens. Some colors will be perceived as sharp at the focus point of the lens, while others will not. The unfocused colors may form a fuzzy ghost image around the focused image.

As will be explained in more detail in the description of the invention, chromatic aberration may occur in a human eye, which, like inanimate optical systems, includes light-refracting elements. The structure of the eye is schematically illustrated in FIG. 4. Among the major parts of the eye are a cornea 2, an iris 4, a retina 6, an eye crystalline lens 8, a ciliary body 10, and ciliary zonules 12.

The cornea 2 is a transparent membrane that protects the eye from the outside world while allowing light to enter the eye. The iris 4 controls the amount of light that enters the eye by opening or closing a pupil, the variable aperture of the eye. The variations in the size of the pupil allow the eye to function over a wide range of light intensities. Thus, the pupil contracts to limit the amount of light in a bright environment, and fully opens in a dim light. The pupil also contracts for near vision, increasing the depth of field to improve perception of objects located in close proximity to the eyes.

The retina 6 is a thin sheet of interconnected nerve cells, which function as detectors, converting information carried by the light (images) into electrical impulses. The detecting elements of the retina 6 include rods and cones. The cones function primarily in normal lighting condition, while the rods are most effective in dim lighting. The sensitivity of the retina is different for different wavelengths within the visible range. The retina is most sensitive in the middle of the visible range, specifically in the green/yellow color sub-ranges, and least sensitive at both ends of the visible range, namely in the red and blue sub-ranges. The spectral sensitivity is also different for rods and cones. Thus, the peak of spectral sensitivity in normal lighting conditions (cone vision) is approximately 555 nm. In dim lighting (rod vision), the peak of sensitivity is approximately 510 nm. The retina is connected to the optic nerve that carries the information gathered by the eye to the brain. When light enters the eye, the crystalline lens 8 projects an inverted image on the retina 6.

The crystalline lens 8 is a transparent convex-shaped structure that focuses the light entering the eye to form a clear image on the retina 6. If the focus point of the crystalline lens 8 is on the retina 6, the perceived image is sharp. If the focus point is in front of or behind the retina, the sharpness of the image may suffer. The phenomenon of chromatic aberration observed in the inanimate optical systems also occurs in the eye. Nevertheless, in most circumstances, all colors are perceived as sharp to an observer because of various compensating mechanisms of the eye.

The crystalline lens 8 is attached to the ciliary body 10 by way of the ciliary zonules 12. The ciliary body 10 contains a ciliary muscle. The eye crystalline lens 8, the ciliary body 10, and the ciliary zonules 12 work together to keep the images entering the eye in focus.

The ability of the eyes to focus clearly on a target of interest at any distance is called accommodation. It is one of the most important visual skills. Although the thin lens equation (1/S+1/S′=1/f) applies to ideal inanimate lenses, its general principles are helpful to describe the accommodation function of the eye. With respect to the thin lens equation, the focusing power of the eye is 1/f, the distance to an observed target is S, and the distance from the eye lens to the image of the target is S′. As described, an image is sharp if it is focused on the retina. The distance between the crystalline lens and the retina is essentially constant. Thus, the distance S′ between the crystalline lens and the image must also be kept essentially constant regardless of the target distance S, which continuously changes as a function of the environment. Applying the thin lens equation, the term 1/S′ remains constant, the term 1/S is changing, and therefore, the term 1/f must change with the change in the distance S to maintain the sharpness of the image. The essential mechanism of accommodation therefore involves changing the focusing power of the eye. The smaller the distance to the observed target, the greater the required focusing power of the eye.

A normal eye does not require any increase in the focusing power in order to clearly see a target at 20 feet or beyond. The table below illustrates a useful non-limiting example of the relationship between the distance from an eye to a target of observation and the required focusing power for a normal eye (in diopters):

| | Required focusing |
|---|---|
| Distance | power of a normal eye |
| (inches) | (diopters) |
| 40 | 1.0 |
| 26 | 1.5 |
| 20 | 2.0 |
| 16 | 2.5 |
| 13 | 3.0 |

Referring to FIG. 4, the change in the focusing power of the eye lens 8 is accomplished by changing the shape of the lens 8 with the help of the ciliary body 10 and the ciliary zonules 12. If the observed target moves closer, the ciliary muscle of the ciliary body 10 constricts thereby causing the zonules 12 to slacken and allowing the crystalline lens 8 to bulge. The resulting increase in the convex cross-section of the crystalline lens 8 increases its focusing power. If the observed target moves away from the eye, the ciliary muscle relaxes, tightening the zonules 12, and flattening the lens 8, thereby reducing the focusing power of a normal eye. At the distance of more than 20 feet, the ciliary muscle is usually relaxed.

In addition to accommodation, other essential visual skills include fixation (the ability to accurately aim the eyes at a target of interest), saccadics (the ability of the eyes to move accurately, efficiently, and rapidly from one target of interest to another), and binocular vision (the ability of the eyes to work together as a team). In large part and for a large proportion of people, inefficiency in any of these essential skills results in visual fatigue and stress associated with visually oriented tasks. It may become difficult for the eyes to aim, move and focus while working as a team, causing discomfort, loss of productivity, and less than optimal educational and/or occupational performance in general. Furthermore, the stress created by the inefficient function of these skills may contribute to the development of eyesight related problems (i.e., myopia, astigmatism). Summarizing, inefficiency in any of the essential visual skills may cause discomfort, loss of productivity, and less than optimal educational and/or occupational performance in general.

To optimize visual functioning and hopefully prevent visual deterioration, the visual system (the eyes, eye muscles and brain centers associated with vision) can be trained to work more efficiently. Vision is a skill that can be trained. The benefits of eye training are multidimensional. Among the benefits, training the eyes provides a physiological improvement in the responsiveness of the entire visual system. The eye muscles, for example, like all trainable muscles improve when properly trained. In effect, they benefit from eye training just as different, more visible human muscles benefit from other forms of exercise.

It is known that physical training improves the ability of the muscular and neurological system to respond with greater speed, accuracy, flexibility and fluidity, thereby enhancing overall performance. The same holds true for training the visual skills required for optimal visual performance. Most of the changes that take place as a function of physical training are gradual and occur over an extended period of time. The same holds true for the eyes. They adapt optimally to exercise that moderately exceeds their capacity.

Therefore, there is a continued and important need for new eye exercise systems and methods. Particularly, there is a need for eye exercise systems and methods that can be used in a variety of locations; use moderate levels of exercise, and that may be used to train a variety of visual functions simultaneously.

### SUMMARY OF THE INVENTION

The present invention addresses these needs by providing eye exercise devices and methods that use the eye's natural response to different colors to train the eye(s). In accordance with one aspect, the invention provides an eye exercise device that includes: a) a housing, including a plurality of colored light sources viewable by an observer and disposed in a substantially linear alignment, the colored light sources being of at least two different colors, including a first color which causes the eye to increase the focusing power of the eye to gain a sharp image of the first color, and a second color which causes the eye to decrease the focusing power of the eye to gain a sharp image of the second color; and b) a controller for controlling the display of the light sources to an observer.

Preferably, the light sources of the first color are mounted in an alternating arrangement with the light sources of the second color. Preferably, the first color is selected from the group consisting of orange and red, and the second color is selected from the group consisting of violet, indigo, turquoise, and blue. The more preferred first color is red, and the more preferred second color is blue or violet. The preferred light sources are light emitting diodes.

The device may further include eyeglasses having interchangeable red and blue or violet filters for selectively affecting the display of the light sources. The device may also further include a control panel for adjustment of the controller.

In accordance with one embodiment, the housing is a horizontal bar, and the eye exercise device further includes a handle connected between two ends of the horizontal bar, dividing the horizontal bar into two segments, each of the segments extending from one of the ends of the horizontal bar to the location where the handle is connected. The horizontal bar has a top surface and a bottom surface. The top surface houses the light sources. The top surface of the horizontal bar may also include a linear marking extending substantially between the ends of the horizontal bar. The handle is connected to the horizontal bar from the bottom surfaces side. The preferred shape of the handle allows placement of the device in a vertical, oblique, or horizontal position with respect to a horizontal plane without additional structural elements. The preferred shape of the handle is octagonal. Also, preferably, at least one of the ends of the horizontal bar defines an open recess that is used in some of the eye exercises.

In a more preferred embodiment, the horizontal bar is foldable so that the eye exercise device may be placed in an operational position, in which the horizontal bar is substantially perpendicular to the handle, or a storage position in which the horizontal bar is folded and the two segments of the bar are substantially parallel with and laying adjacent to the handle. Preferably, the location where the handle is connected to the horizontal bar is substantially equidistant from both ends of the horizontal bar. Preferably, the light sources are also substantially equidistant from each other.

In accordance with another aspect, the invention provides an eye exercise device that includes a) one or more first light sources of a first color that causes the eye to increase the focusing power of the eye to gain a sharp image of the first light sources, b) one or more second light sources of a second color that causes the eye to decrease the focusing power of the eye to gain a sharp image of the second light sources, the second color being different from the first color, c) a housing to which the first and second light sources are mounted, and d) a programmable controller to alternate the display of the first and second light sources to exercise one or more eyes of a person by alternately causing an increase and decrease in the focus power of an eye of a human subject observing the light sources.

Preferably, the first color is selected from the group consisting of orange and red, and the second color is selected from the group consisting of violet, indigo, turquoise, and blue. The preferred first color is red, and the second color is blue or violet. In this aspect, the eye exercise device may include any of the specific features previously described above in reference to another device aspect of the invention.

According to another aspect, the invention provides a method of exercising an eye of a person that includes a) exposing the observer to a predetermined arrangement of (i) one or more first light sources of a first color that causes the eye to increase the focusing power to gain a sharp image of the first light sources, and (ii) one or more second light sources of a second color different than the first color that causes the eye to decrease the focusing power to gain a sharp image of the second light sources; and b) alternating the display of the first and second light sources to exercise the eye of the observer observing the light sources by alternately causing the focusing power to increase and decrease.

Preferably, the alternating includes alternating the display between the first color being selected from the group consisting of orange and red and the second color being selected from the group consisting of violet, indigo, turquoise, and blue. The preferred first color is red, and the preferred second color is blue or violet. The preferred pre-determined arrangement is a substantially linear alignment of the light sources.

In accordance with this aspect of the invention, the method further includes positioning the observer vertically in front of the substantially linear alignment of the light sources during the exercise. Preferably, the light sources and the eyes of the observer are at approximately the same level. The observer may wear eyeglasses having interchangeable red and blue or violet filters to selectively affect the display of the light sources to the observer.

In one embodiment of this aspect of the invention, the method further includes placing the light sources in such a manner that a vertical plane containing the substantially linear alignment of the light sources and a vertical plane containing an imaginary line drawn through the eyes of the observer are substantially parallel to each other. The substantially linear alignment of the light sources may be placed in a horizontal, oblique, or vertical position with respect to a horizontal plane containing the eyes of the observer. Once the observer and the light sources are situated as desired, the observer is exposed to a discreet exercise sequence. Thereafter, the distance between the observer and the light sources may be changed, and the observer may be exposed to another discreet exercise sequence. During the exercise, the light sources are preferably activated consecutively and one at a time.

In another embodiment of this aspect of the invention, the method further includes placing the light sources in such a manner that a vertical plane containing the substantially linear alignment of the light sources and a vertical plane containing an imaginary line drawn through the eyes of the observer are substantially perpendicular to each other. Preferably, the method further includes activating the light sources consecutively and one at a time.

In accordance with another aspect, the invention provides a method of exercising an eye or eyes of an observer, including a) exposing the observer to a plurality of red and blue light sources, and b) activating one or more of the light sources to display the light sources to the observer one-at-a-time.

Preferably, the light sources are in a substantially linear alignment. Also, the red light sources and the blue light sources are preferably mounted in an alternating arrangement with each other. In the preferred embodiment, the light sources are displayed sequentially.

In accordance with another preferred aspect, the invention provides a kit for exercising eyes including a) a device that includes a plurality of colored light sources viewable by an observer and disposed in a substantially linear alignment, the colored light sources being of at least two different colors, including a first color which causes the eye to increase its focusing power to gain a sharp image of the first color and a second color which causes the eye to decrease its focusing power to gain a sharp image of the second color; and b) eyeglasses having interchangeable color filters of the first color and second color for selectively affecting the display of the light sources to the human subject.

Preferably, the light sources of the first color are mounted in an alternating arrangement with the light sources of the second color. Preferably, the first color is selected from the group consisting of orange and red, and the second color is selected from the group consisting of violet, indigo, turquoise, and blue. The more preferred first color is red, and the more preferred second color is blue or violet.

In another aspect, the invention provides a system for exercising an observer's eyes, the system including a display device; a computer processor with associated computer memory, the processor operatively coupled to the display, the computer memory, and one or more input devices; the computer processor being configured to display on the display device a predetermined sequence of colored images, the color of each colored image having varying wavelength, each successive colored image having a sufficiently different wavelength from its predecessor color image to cause the focusing power of the observer's eyes to adjust, each colored image being singly displayed for a period of time sufficient for the focusing power of an observer's eyes to adjust. Preferably, the system is further configured to display each colored image on the display device to the observer in a perceived location for a period of time sufficient for the observer's eyes to adjust their focusing power; move the colored image to a different perceived location for a period of time sufficient for the observer's eyes to again adjust their focusing power; and repeat the movement of the colored image multiple times for a given period of time sufficient to exercise the observer's eyes. The preferred color of the colored images is selected to alternate between a first color and a second color, wherein the first color has a spectral wavelength longer than the wavelength of the eye's peak spectral sensitivity in normal lighting conditions, and the second color has a spectral wavelength shorter than the wavelength of the eye's peak spectral sensitivity. In one embodiment, the system further includes a network of one or more computers configured as a source of information for configuring the computer processor; and a network adapter associated with the computer processor, the network adapter providing at least one communication channel connecting the computer processor to the network of computers, wherein the computer processor is configured with the information communicated from the network of computers.

In yet another aspect, the invention provides a medium having stored thereon instructions for directing a display device observed by an observer to display a sequence of colored images for exercising the observer's eyes, the instructions causing the display device to display a sequence of colored images on the display device, each image having a color varying from the color of the previous image, and each image being displayed for a period of time sufficient for the focusing power of the observer's eyes to adjust. The medium according may be computer-readable, the instructions may be computer-implemented, and the display device may be computer-controlled. Preferably, the medium is internal to a computing device.

In yet another aspect, the invention provides a computer-implemented method for exercising an observer's eyes through observation of colored images on a display device, by way of a computer-implemented program, a predetermined sequence of colored images, the color of each colored image having varying wavelength, each successive colored image having a sufficiently different wavelength from its predecessor color image to cause the focusing power of the observer's eyes to adjust, each colored image being singly displayed for a period of time sufficient for the focusing power of an observer's eyes to adjust. Preferably, in the computer-implemented method according to this aspect, the color of the colored images is selected to alternate between a first color and a second color, wherein the first color has a spectral wavelength longer than the wavelength of the eye's peak spectral sensitivity in normal lighting conditions, and the second color has a spectral wavelength shorter than the wavelength of the eye's peak spectral sensitivity.

In yet another aspect, the invention provides a method of exercising observer's eyes by displaying to the observer a predetermined sequence of colored objects that includes at least a first colored object and a second colored object, wherein the color of the second colored object is different from the color of the first colored object; the colors of the first colored object and the second colored object having spectral wavelengths on different sides of peak spectral sensitivity of a human eye in normal lighting conditions. In one non-limiting alternative, the color of the first colored object has a spectral wavelength longer than the wavelength of the eye's peak spectral sensitivity and the color of the second colored object has a spectral wavelength shorter than the wavelength of the eye's peak spectral sensitivity. In another non-limiting alternative, the color of the first object has a spectral wavelength shorter than the wavelength of the eye's peak spectral sensitivity and the color of the second colored object has a spectral wavelength longer than the wavelength of the eye's peak spectral sensitivity. Non-limiting examples of the colors of the first and second colored objects include red and blue, blue and red, red and indigo, and indigo and red.

The predetermined sequence may further include a third object having color different than the color of the second colored object. Preferably, the color of the third colored object is located on a different side of the peak spectral sensitivity from the color of the second colored object. The predetermined sequence may include a plurality of colored objects greater than three. Preferably, each colored object in the predetermined sequence is displayed one at a time for a period of time sufficient for the focusing power of the observer's eyes to adjust. The colored objects may be images produced on a display device, a real physical object or any other object perceptible by a human eye. Preferably, the first colored object consists only of a single color and the second colored object consists only of a single color. The shapes and locations of the colored objects in the predetermined sequence may be varied, if desired. In the method aspects of the invention, the eye exercise devices described herein may be used. The features, embodiments, or aspects of the eye exercise devices are suitable for use with the methods of the invention.

It is understood that both the foregoing general description and the following detailed description are exemplary and are intended to provide further explanation of the invention claimed.

### DESCRIPTION OF THE DRAWINGS

FIG. 1 illustrates characteristics of lenses, such as focus point and focal length;

FIG. 2 illustrates focusing of object images by lenses;

FIG. 3 illustrates chromatic aberration in inanimate lenses;

FIG. 4 is a schematic cross-sectional view of a human eye;

FIG. 5 illustrates longitudinal chromatic aberration in a human eye;

FIG. 6 shows an approximation of the relative spectral sensitivity curve of the retina in normal lighting conditions;

FIGS. 7A-7B illustrate adjustment of eye's focusing power due to chromatic aberration;

FIGS. 8A-8B show an eye exercise device in accordance with the preferred aspect of the invention;

FIG. 9A-9B illustrate examples of eye exercises in accordance with one embodiment of the invention;

FIGS. 10A-10B show a preferred embodiment of the eye exercise device in accordance with the invention;

FIG. 11 is a block functional diagram of the eye exercise device in accordance with the preferred embodiment of the invention;

FIGS. 12A-12E illustrate examples of exercises with the eye exercise device of the preferred embodiment of the invention.

FIG. 13 is a flowchart depicting an exemplary process for exercising a user's eyes by changing the color of a displayed image over time in accordance with a preferred embodiment of the present invention;

FIG. 14 is a flowchart of an exemplary process for exercising a user's eyes by changing the perceived location of a displayed image over time in accordance with a preferred embodiment of the present invention;

FIG. 15 is a flowchart of an exemplary process for exercising a user's eyes by changing both the perceived location and the color of a displayed image over time in accordance with a preferred embodiment of the present invention;

FIG. 16 is a flowchart of an exemplary animation and display sequence in accordance with the preferred embodiment of the present invention;

FIG. 17 is an exemplary main menu screen of a user interface display for an eye exercise application in accordance with a preferred embodiment of the present invention;

FIG. 18 is an exemplary color/shape sequence selection screen of a user interface display for an eye exercise application in accordance with a preferred embodiment of the present invention;

FIG. 19 is an exemplary location/movement selection screen of a user interface display for an eye exercise application in accordance with a preferred embodiment of the invention;

FIGS. 20(a), 20(b), 20(c), and 20(d) depict an exemplary eye exercise display color change sequence in accordance with a preferred embodiment of the invention;

FIG. 21(a), 21(b), 21(c), and 21(d) depict an exemplary eye exercise display image location change sequence in accordance with a preferred embodiment of the invention;

FIG. 22(a), 22(b), 22(c), and 22(d) depict an exemplary eye exercise combined display image color and location change sequence in accordance with a preferred embodiment of the invention;

FIG. 23(a), 23(b), 23(c), and 23(d) depict an exemplary eye exercise three-dimensional display image perceived depth and location change sequence in accordance with a preferred embodiment of the invention;

FIG. 24 illustrates examples of eye exercise sequences in accordance with an embodiment the invention;

FIG. 25 illustrates examples of eye exercise sequences in accordance with an embodiment the invention;

FIG. 26 is a depiction of a sample head mounted unit for use in a preferred embodiment of the invention; and

FIGS. 27(a), 27(b), 27(c), 27(d), and 27(e) depict a user performing assorted eye exercises utilizing a display device in accordance with a preferred embodiment of the present invention.

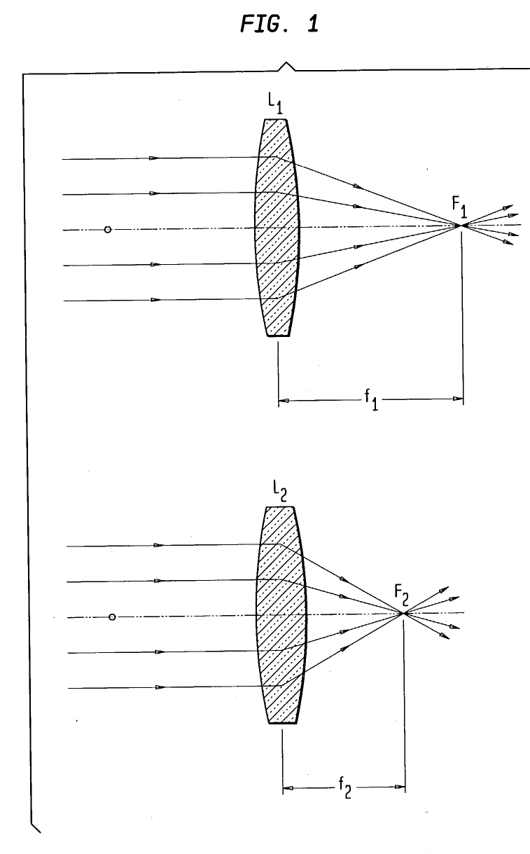

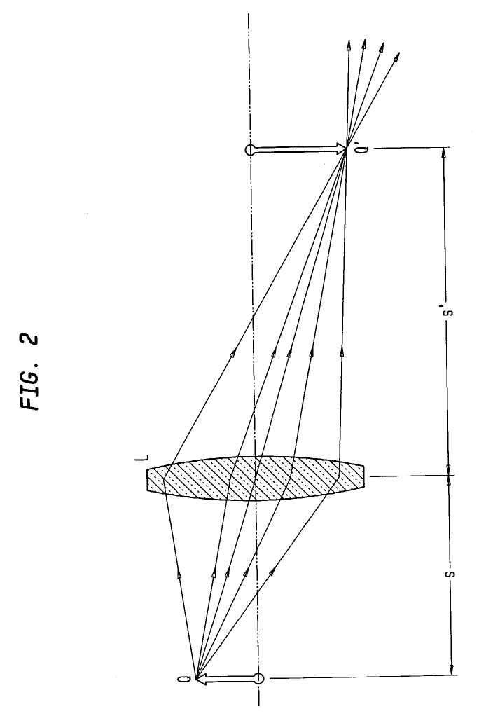

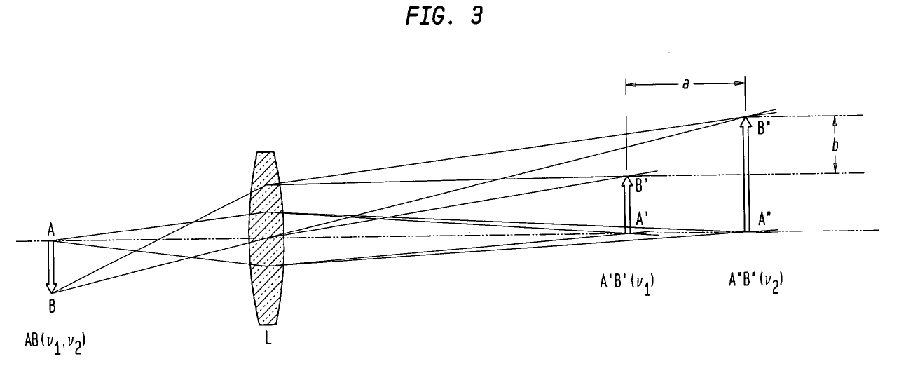

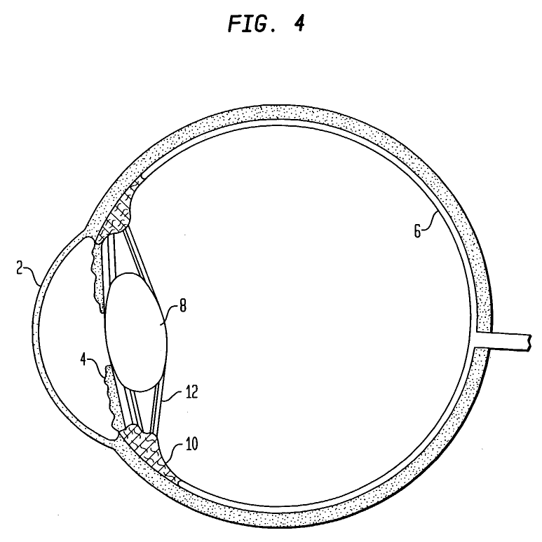

### DESCRIPTION OF PREFERRED EMBODIMENTS

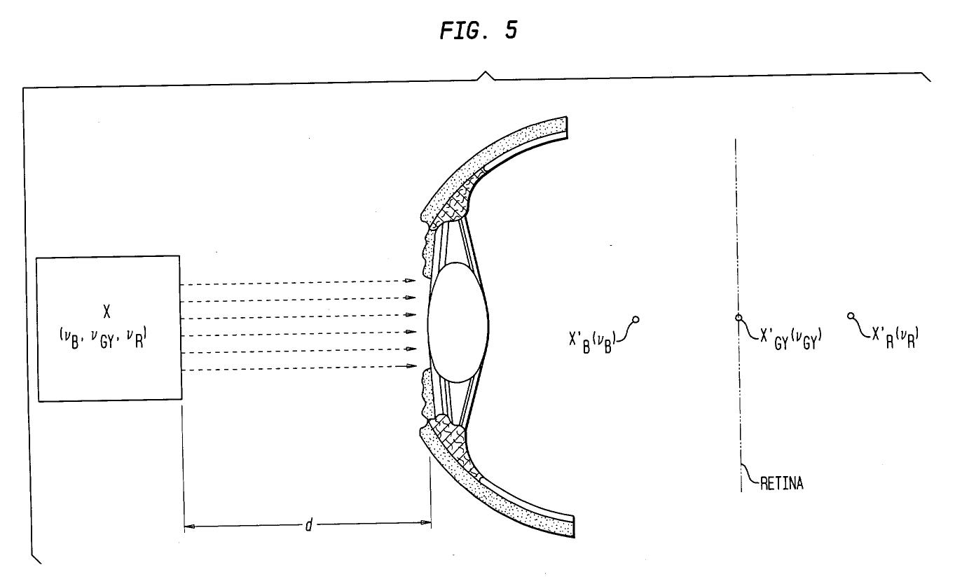

While the invention is by no means limited to any specific theory, the inventor recognized that chromatic aberration of the eye might be utilized in exercising the eye(s). Suppose, the eye observes an object X having a full color spectrum (FIG. 5). The object X reflects or emits light waves of substantially all wavelengths of the visible range, including a light wave of the wavelength ν_{B} in the blue color sub-range (the light wave ν_{B}), a light wave of the wavelength ν_{GY} in the green-yellow color sub-range (the light wave ν_{GY}), and a light wave with the wavelength V_{R} in the red color sub-range (the light wave ν_{R}). Because of the different refractive indexes (n(ν_{B})>n(ν_{GY})>n(ν_{R})), the light of longer wavelengths (e.g., ν_{R}) penetrates deeper into the eye than the light of shorter wavelength (e.g., ν_{B}). The light waves ν_{B}, ν_{GY}, and ν_{R} focus as images X′_{B}, X′_{GY}, and X′_{R}, respectively, at different distances from the eye lens, resulting in a longitudinal chromatic aberration of the eye lens.

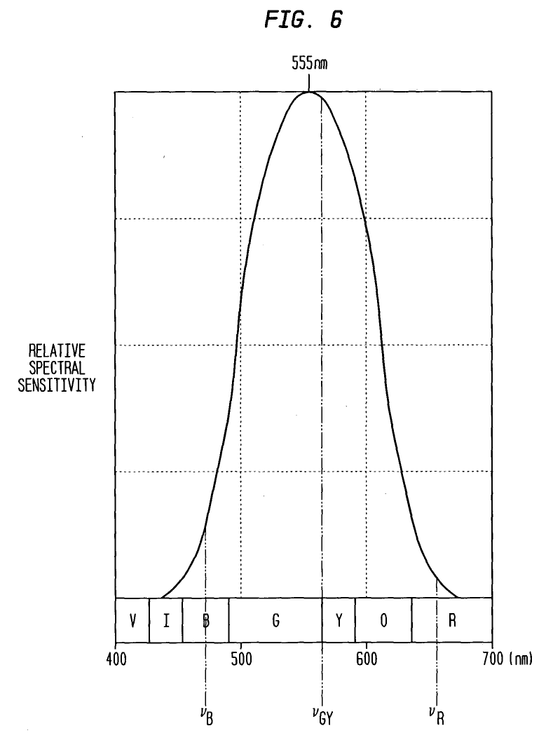

As seen in FIG. 5, the optimal focusing powers for the light waves ν_{B}, ν_{GY}, and ν_{R} are different because of the longitudinal chromatic aberration. The blue image X′_{B}, the green-yellow image X′_{GY}, and the red image X′_{R} cannot be focused on the retina at the same time. For the full color object X, the eye adjusts its focusing power to focus the light wave for which the retina has greatest spectral sensitivity. FIG. 6 shows the relationship between the wavelength and sensitivity of the retina (the relative spectral sensitivity curve) in normal lighting conditions (V denotes violet, I denotes indigo, B denotes blue, G denotes green, Y denotes yellow, O denotes orange, and R denotes red color sub-ranges). Referring to FIG. 6, the sensitivity of the retina for the light wave ν_{GY} is dramatically greater than for the light waves ν_{B} and ν_{R}. In other words, the retina detects substantially more light at the wavelength ν_{GY} than at the wavelengths ν_{B} or ν_{R}. For this reason, while observing the full color spectrum object X, the eye adjusts the focusing power to focus the image X′_{YB} on the retina (FIG. 5). The blue image X′_{B} focuses in front of the anterior surface of the retina, and the red image X′_{R} focuses behind the anterior surface of the retina.

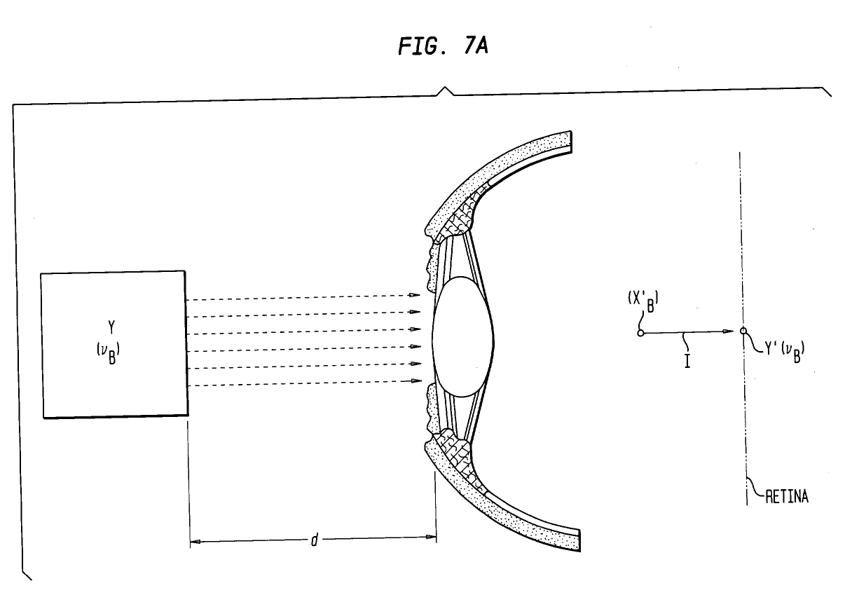

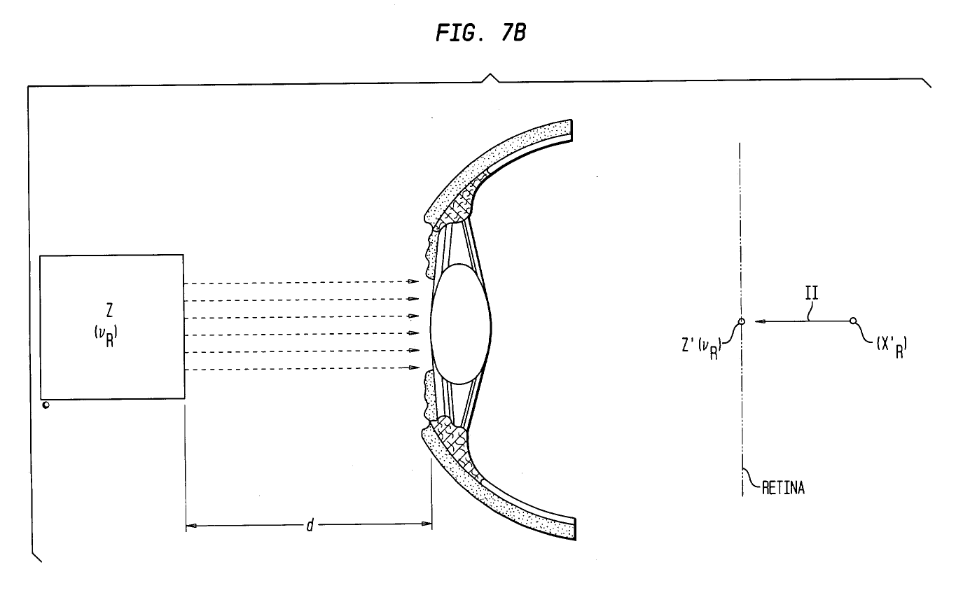

Suppose, the object X is replaced with an object Y that emits or reflects only the blue light wave ν_{B}, producing a blue image Y′ (FIG. 7A). It is no longer necessary to maintain the focusing power that was suitable for the object X. At the focusing power optimal for the object X, the blue image (X′_{B}) was located in front of the retina. To focus the image Y′ on the retina, the eye decreases the focusing power relative to the focusing power for the object X (shown by the arrow I). If instead of the blue object Y, the object X is replaced with a red object Z (FIG. 7B) that emits or reflects only the red light wave ν_{R}, the eye increases the focusing power relative to the focusing power for the object X (shown by arrow II). The adjustments in the focusing power are believed to occur automatically.

Thus, in accordance with a preferred aspect of the invention, the eye may be exercised by alternate exposure to light of a color(s) that includes the wavelength(s) νₐ, and a color(s) that includes the wavelength ν_{b} shorter than νₐ, where νₐ and ν_{b} are different from each other. Alternate exposure to colors of the different wavelengths νₐ and ν_{b} causes the eye to alternately increase and decrease its focusing power to maintain the sharpness of perception. It is believed that, in response to such alternating exposure, the ciliary muscle acts in the opposite directions, resulting in a gentle rocking motion that moderately exerts and exercises the eye muscles. The focusing and aiming mechanisms of the eye are alternately stimulated and relaxed, training the eye in a natural way without using external lenses and prisms. The neural functions associated with the visual skills, including the brain, are also trained.

The greater the difference between the wavelengths of the first and second colors, the greater is the magnitude of the focusing power adjustment. Therefore, preferably, the difference Δν (νₐ−ν_{b}) between the wavelengths νₐ and ν_{b} is maximized. The greater is Δν, the greater the training effect. The colors of wavelengths close to the peak of spectral sensitivity curve are preferably excluded when the observer is exposed to colors νₐ and ν_{b}.

Preferably, if ν₀ is the wavelength at which a normal eye has a peak of spectral sensitivity in normal lighting conditions, νₐ is longer than ν₀, and ν_{b} is shorter than ν₀. If ν₀=555 nm, the focusing power of a normal eye required to gain a sharp perception of a color with a wavelength of 555 nm in normal lighting conditions at a given distance d may be defined as the mean eye focusing power. The mean focusing power divides the visible range into two color groups for the purposes of the present invention. The first group of first colors include colors that, when observed at the distance d, require the eye to increase the focusing power with respect to the mean focusing power to gain a sharp image of the first colors. The second group (or second colors) include colors that, when observed at the distance d, require the eye to decrease the focusing power with respect to the mean focusing power to gain a sharp image of the second color. Pure monochromatic colors or colors comprising mixtures of wavelengths may be used. Examples of first colors include orange and red. Examples of second colors include violet, indigo, turquoise, and blue. In the methods and devices of the present invention, the preferred first color is red, and the preferred second colors are blue and violet. Red and blue or violet light waves have wavelengths at the opposite ends of the visible light range. For this reason, it is believed that the training effect of alternate exposure to red and blue or violet colors is greater than for other color pairs.

Preferably, an observer is alternately exposed to colors of first and second groups. For example, the observer may be exposed to blue color, followed by red color, followed by blue color, and so on, with the exclusion of the green or yellow colors from the environment and the target of observation. However, the colors with high spectral sensitivity may also be included in the exposure sequence. An example of such sequence is blue, green, red, green, blue, and so on.

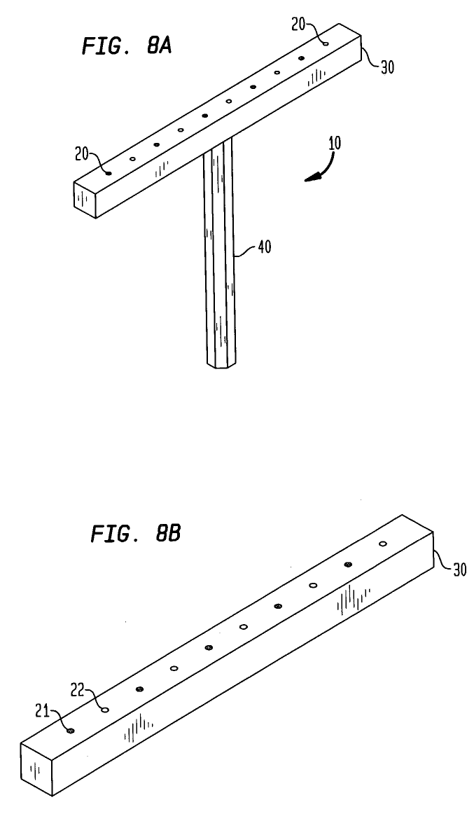

FIGS. 8A and 8B show the preferred eye exercise device in accordance with the present invention. It should be understood that the specific embodiments are described below for the purpose of illustration only. The major components of the device 10 are a plurality of colored light sources 20, a housing 30, and a handle 40 (FIG. 8A). The handle 40 supports the housing 30. Preferably, the handle 40 has squire or octagonal shape. As seen from FIG. 8A, the housing 30 supports or houses the colored light sources 20 in a substantially linear alignment. Other arrangements of the light sources are also possible although the linear alignment is preferred.

The colored light sources 20 preferably include light sources 21 of the first color(s), and light sources 22 of the second color(s) (FIG. 8B). The preferred first color is red, and the preferred second color is blue or violet. The preferred light sources are light emitting diodes (LEDs).

Preferably, the light sources 21 and 22 are arranged in an alternating pattern to each other. Non-limiting examples of such patterns are shown in the table:

| Color(s) | | | |
|---|---|---|---|
| of the | | Total | |
| light | Color(s) of | number of | |
| sources | the light | light | |
| 21 | sources 22 | sources | Pattern* |
| R | B | 12 | R, B, R, B, R, B, R, B, R, B, R, B |
| R | V | 6 | V, R, V, R, V, R |
| R | B, V, T, I | 10 | R, V, R, I, R, B, R, T, R, V, R, B |
| R | B | 9 | B, R, B, R, B, R, B, R, B |

*R denotes red, B denotes blue, V denotes violet, T denotes turquoise, I denotes indigo.

In operation, the subject/observer is placed in front of the device 10, with the device 10 set up in a desired orientation with respect to the observer. For example, the light sources 20 may be placed at, above or below the eye level of the observer, or at an angle to the eyes of the observer. Also, the device 10 may be set up with the colored light sources 20 located horizontally, vertically and/or obliquely relative to the observer. The device housing 30 of the device 10 may also extend perpendicularly away from the observer's nose.

Then, the person controlling the device 10 (e.g., the observer) activates the device, selects the exercise program, and initiates the desired exercise. During the exercise, one or more of the plurality of colored light sources 20 are illuminated for display in the manner selected by the user, for example, sequentially left to right and back right to left, sequentially right to left, randomly, and so on. The light source is “displayed” when it is actuated (turned on) at a given moment of time. The colored light sources 20 may be displayed simultaneously, one at a time, or in other desired ways and sequences. Preferably, the light sources 20 are displayed sequentially one at a time. More preferably, the first light sources are displayed alternately with the second light sources. For example, a blue light source is displayed, followed by a red light source, followed by a blue light source, and so on. The light sources 20 are arranged in an alternating pattern, and therefore sequential, one-at-a-time display alternately displays light sources 21 and 22. In accordance with the preferred embodiment, during the eye exercise, the subject observes and focuses on each light source as it is displayed.

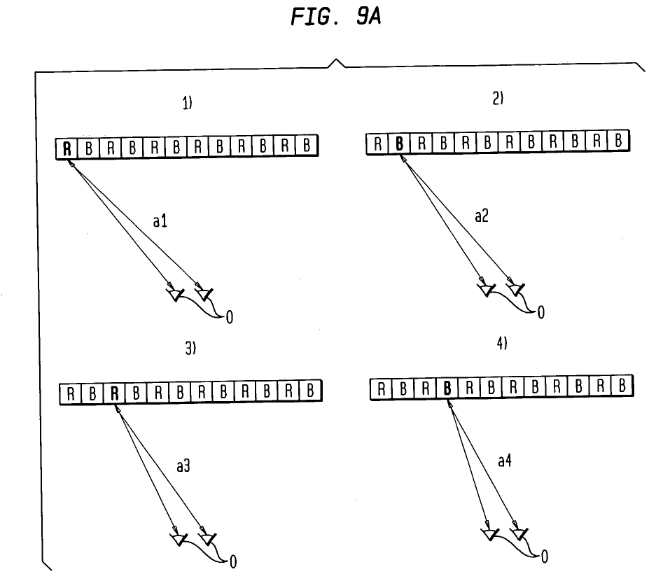

FIGS. 9A and 9B illustrate non-limiting examples of the training exercises with the device having six red and six blue light sources arranged in a R,B,R,B,R,B,R,B,R,B,R,B pattern. O denotes the observer, and the displayed light sources are shown in bold. In the exercises illustrated in FIG. 9A, the light sources are set up in a plane parallel to the observer's eyes, and displayed one-at-a-time from left to right. At the time 1, the observer perceives a red light source at a distance a1, at the time 2, a blue light source at a distance a2, at the time 3, a red light source at a distance a3, and so on. Thus, both the color and the distance to the target of observation (the displayed light source) change during the exercise. As described, the eye adjusts its focusing power in response to both change in color and distance. The location of the displayed light source in the horizontal plane relative to the observer is also changing, exercising the ability of the observer's eyes to move freely and accurately in the horizontal plane as the eyes track the movement of the displayed light source.

In the exercise shown in FIG. 9B, the light sources are placed perpendicularly to the observer. At the time 1, the observer perceives a red light source at the distance b1, at the time 2, a blue light source at the distance b2, and so on. As in the exercise shown in FIG. 9A, both the color of the displayed light source and the distance change. The change in the distance (e.g., from b1 to b2) is larger. In this exercise, the eyes also converge more or less as the target of observation moves closer or further, exercising the ability of the eyes to work together as a team. The use of different exercises available with the device 10 allows the simultaneous training of a variety of different visual skills under different conditions.

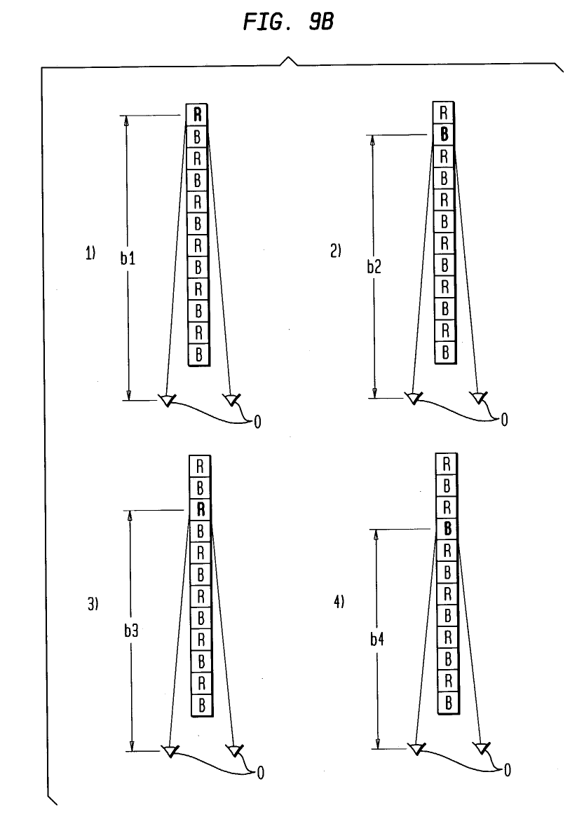

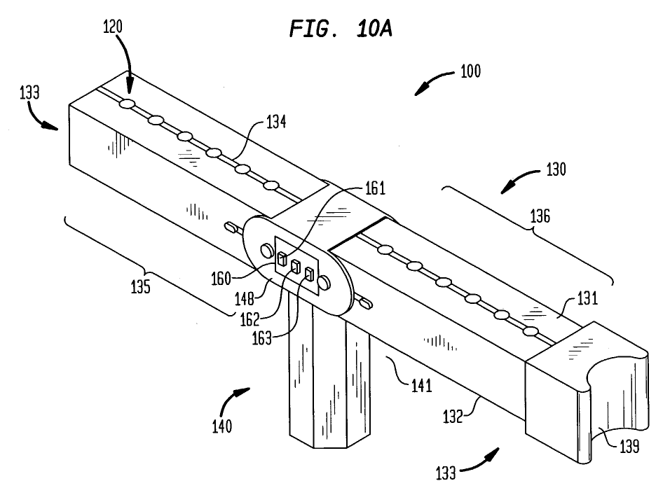

In the preferred embodiment, the invention provides a portable eye exercise device 100 shown in FIGS. 10A-10B. The device 100 is foldable for convenient use, and may be used at home, while traveling, and the like. The device 100 is intended primarily for personal use, without professional assistance.

As seen from FIG. 10A, the device 100 includes a plurality of LEDs 120, a foldable horizontal bar 130, a handle 140, a control panel 160, a display panel 169 (not shown), and a controller 170 (not shown). The horizontal bar 130 has a top surface 131 and a bottom surface 132 (FIG. 10B). Red LEDs 121 and blue LEDs 122 are mounted on the top surface 131 in an alternating arrangement. Each LED may be referred to using numbers from (1) to (12). A linear stripe 134 extends between ends 133 of the horizontal bar 130. One of the ends 133 defines a recessed bridge 139, which is used in some eye exercises to ensure appropriate position for the person using the device 100. A proximate end 141 of the handle 140 is connected to the bar 130 at a connection location 148, which divides the bar 130 into a right segment 135 and a left segment 136. When the device 100 is used for eye exercises, both segments are unfolded (FIG. 10A). If the device 100 is not in use, the segments 135 and 136 may be folded along the handle 140 for easy storage.

In a preferred variant, the device 100 is a compact, hand-held unit. For instance, the horizontal bar may be 36″ long, the handle may be 4″ long and the LEDs are located 2.75″ apart. The handle may be in the octagonal or other similar form that allows placement of the device in horizontal or vertical orientation without additional support or attachments. When folded for storage, the device is 15-16″ in length and 5-6″ thick. The size of the device may be further minimized if desired.

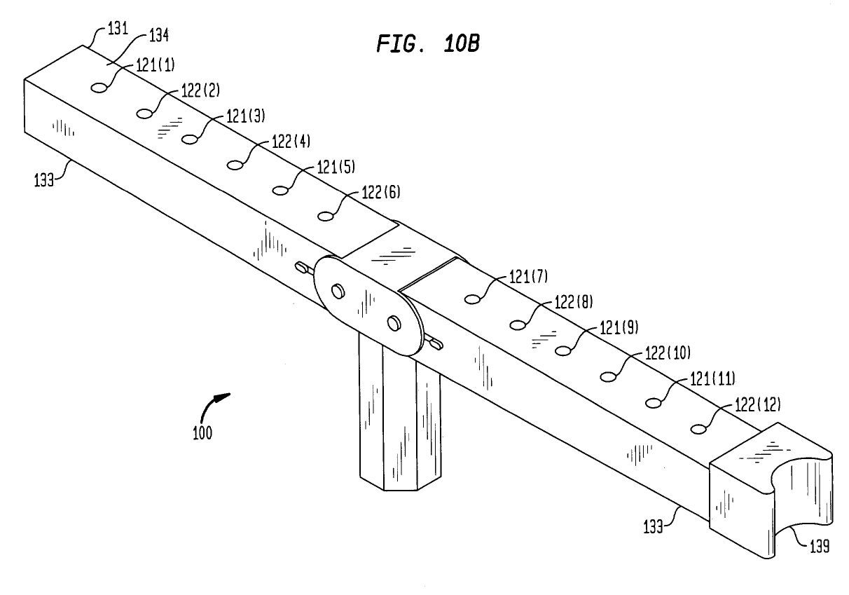

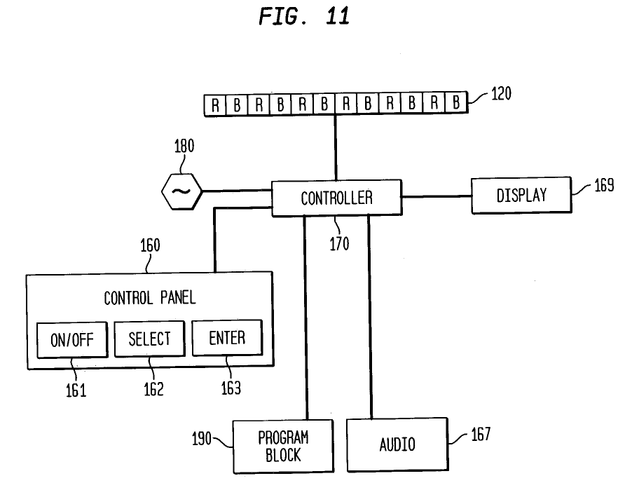

FIG. 11 shows a functional block diagram of the device 100. The controller 170 guides the manner and order of display of the LEDs 120. The controller 170 may be mounted within the horizontal bar 130 or any other portion of the device 100. The LEDs 120 are connected to a source of power 180 through the controller 170. The controller 170 is also connected to the control panel 160, a program block 190, a display 169, and an audio signaling device 167. The controller 170 can comprise a special purpose controller or a general-purpose microprocessor programmed to control the function of the device 100. Any connections, blocks and/or components known in the art may be used to effect the operation of the device 100.

The program block 190 can comprise a memory, which stores instructions for execution by the controller 170, including various pre-set exercise sequences. The display 169 displays the status of an exercise, speed setting, pre-set exercise ID, and the like. For example, the display 169 can comprise an LED screen. An audio signaling device 167 can also be provided to provide the user with information about the progress of the exercise, e.g., start, stop, type, speed, etc.

The control panel 160 is used to operate the device. The control panel 160 preferably has three control buttons: an on/off button 161, a select button 162, and an enter button 163. The on/off button 161 is used to manually turn the device 100 on or off. In one version of the device 100, if an exercise program is not started within a pre-determined time after the device is turned on, the device automatically shuts itself off. The select button 162 allows the user to choose an exercise program and is used to switch between the device functions. The device functions may include selection of the exercise program, setting the speed of the exercise, choosing an auditory feedback options, etc. The enter button 163 is used to operate the selected functions. The functions of the buttons may be altered in any manner known in the art.

The device 100 may store a variety of pre-set actions, operations or exercise programs. For example, the pre-set operations may include certain audio signals to indicate the end or the beginning of an exercise sequence, the display of an LED, a pause between exercises, display sequences for the LEDs 120 selectable by a user, and so on.

The device 100 may provide pre-determined preset speed settings. A speed setting can measure how long a single LED stays displayed or how fast the next LED is displayed. Depending on the speed setting, a given exercise sequence may be done different number of sequence cycles within a pre-determined exercise time (e.g., in the allotted one and one half minute, the Sequence Program I may be done one, two, three or more times depending on the speed setting). The table illustrates the device 100 that may have multiple speed settings, showing the display times for a single LED at each speed setting:

| | Time of display for |
|---|---|
| Speed | a single LED in a |
| setting | sequence (seconds) |
| 0 | 2.5 |
| 1 | 2.0 |
| 2 | 1.75 |
| 3 | 1.5 |
| 4 | 1.25 |
| 5 | 1.0 |
| 6 | .75 |
| 7 | .50 |
| 8 | .25 |
| 9 | .20 |
| C | Changeable speed |
| | setting: each LED |
| | stays on for a |
| | randomly changeable |
| | amount of time. |

The device 100 may be equipped with an auditory feedback option that provides auditory stimulus. The auditory feedback option serves to reinforce the eyes' ability to accurately locate the displayed light source(s). For this purpose, a sound can be generated every time an LED is about to be displayed or concurrently displayed. The sound goes on at the exact moment the LED turns on. Also, the device may beep to indicate the end of the exercise sequence, etc. The may also produce a number of short beeps, for example, followed by one long beep, to indicate that an exercise program is about to begin, etc.

Some of the operations of the device 100 will now be described. Pressing the button 161 on the control panel 160 turns on the device. Once the device had been turned on, a “P” (for program) appears on the LED display 169. By pressing the select button 162 once, a number 1 (for program 1) is displayed on the display. Each time the button 162 is pressed, the display shows the program number associated with the next program. Once the program number of the last program is displayed, the device returns to the program 1.

After the desired program is selected, pressing the enter button 163 causes an “S” (for speed) to come up on the display. The select button 162 is used to set the speed of the device (e.g., the time each LED remains displayed in a sequential, one-at-a-time display of LED's). Initially, the display 169 shows a zero (0), indicating the slowest speed setting. Each successive time the select button 162 is pressed the speed setting advances to the next faster level (e.g., 2,3,4, etc.). Pressing the select button 162 again brings the speed setting back to zero (0).

In general, pressing the button 163 moves the user from program selection to speed selection to auditory feedback selection, etc. Thus, after the speed setting is selected, pressing the enter button 163 causes an “A” (for auditory feedback) to show up on the display 169. By pressing the select button 162 once, a “0” comes up on the display, indicating a “no” for auditory feedback. Pressing the select button 162 a second time causes a number “1” to come up on the display indicating a “yes” for auditory feedback. Pressing the select button one more time brings the auditory feedback setting back to zero (“0”). After selecting no (0) or yes (1) for auditory feedback, the enter button 163 is pressed. The device may now be used in eye exercises.

The above menu system is merely exemplary and other system of menus, icons, displays, etc. can be used for ease of user interaction.

The device 100 may be used for eye movement exercises, which may be performed horizontally, vertically, and in both oblique meridians. In each case, once the device 100 is programmed and oriented in the appropriate meridian, the observer stands or sits in front of the device and presses the enter button 163 to begin the exercise. The device runs the desired exercise program while the user's eyes track the movement of the displayed LEDs. Once proficiency is established, the observer may move closer or further away from the device 100, depending on the desired training effect. As the distance between the observer and the device shortens, the eye movement exercises begin to gently stretch the eye muscles. As the distance increases, the eyes begin developing greater fine-motor control.

The device 100 may also be used to exercise binocular vision while simultaneously providing the user feedback on whether the eyes are working together as a team or not. When a person with normally functioning eyes looks at a target, an area of single binocular vision is created. Points located within this area are seen singly. Points located in front of or behind this area of single binocular vision are perceived as double. This phenomenon is known as physiological diplopia. When a series of fixation targets (e.g., LEDs) are lined up in a straight line moving away from the eyes of the observer with normal binocular vision, the target specifically being viewed appears single while targets in front of and behind appear double. This use of physiological diplopia provides the user visible feedback about their eyes ability to work together as a team. Furthermore, if the fixation targets (e.g., the LED's 120) are connected by a stripe, a viewer with normal binocular vision will also see the appearance of an “X” with the target (LED) being fixated at its intersection. The appearance of an “X”, along with the apparent doubling of the fixation targets (LED's) not being viewed, provides a visible feedback mechanism for the user about the degree to which their eyes work together as a team. This exercise specifically strengthens the user's ability to efficiently use both eyes together as a team during a dynamic situation because the user literally can see when both eyes are being used together and when they are not.

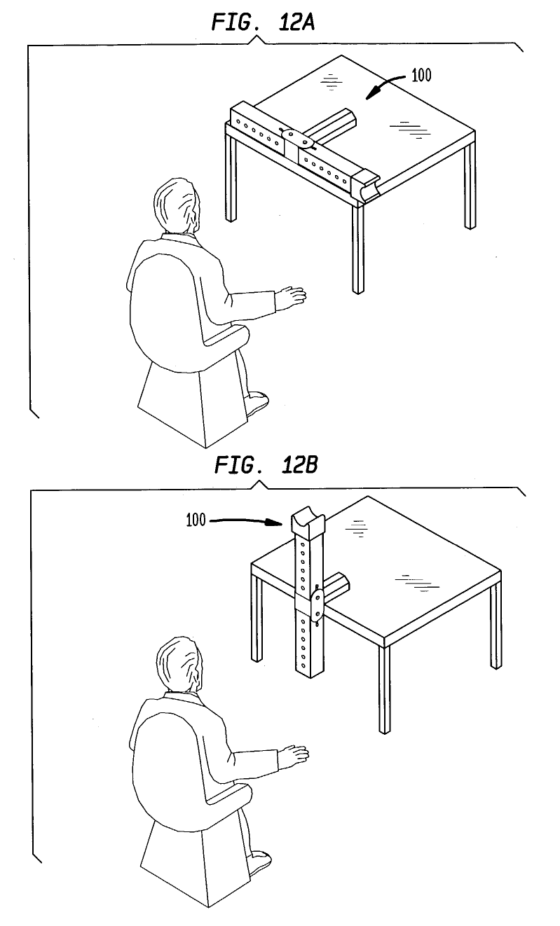

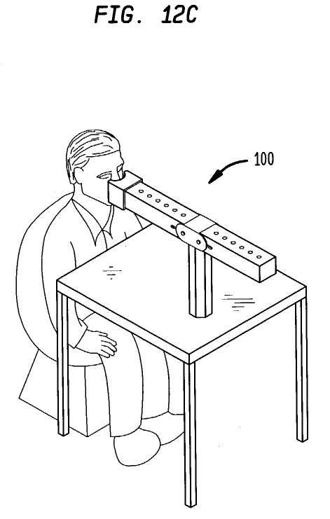

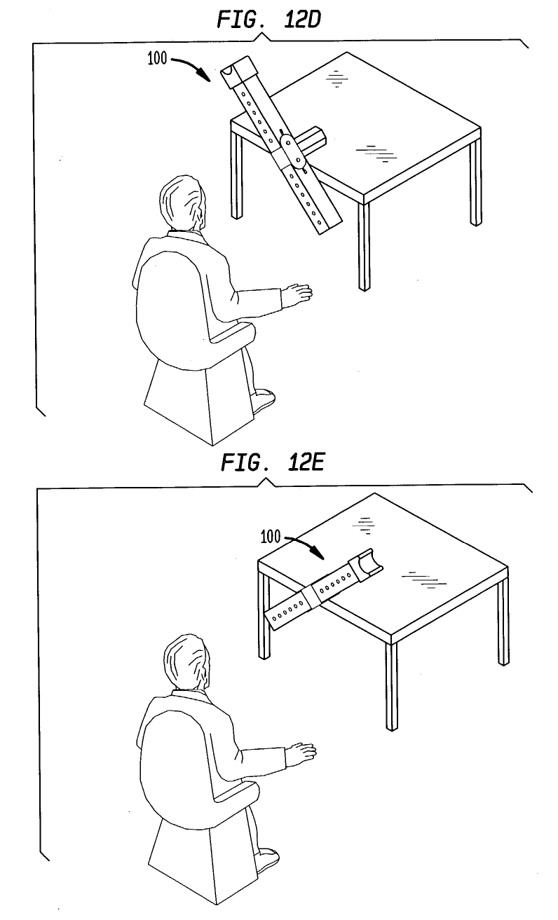

FIGS. 12A-12E illustrate examples of the eye exercises with the device of the invention.

### EXAMPLE 1

### Horizontal Eye Movement Exercises

The device is set up at eye level, oriented for horizontal viewing (FIG. 12A). A chair is placed approximately one yard away from the device 100. The user presses the enter button 163 and sits down in the chair to begin the first eye movement exercise. Once the enter button 163 is pressed, the LED display 169 turns off and begins the auditory countdown to the exercise. For example, if the countdown is 10 seconds long, the device sounds a short beep every second for nine seconds followed by one long beep. The long beep informs the user that an exercise program is about to begin. Once the program begins, the LEDs 120 are displayed from left to right and back from right to left. The user is tracking the displayed LED with the eyes. The purpose of the exercise is to train the user to allow their eyes to move freely and accurately as they track a moving target. The program runs for one and one half minutes and then ends indicating the completion of the first exercise and the beginning of a break period. The user can now relax and gently breathe.

### EXAMPLE 2

### Vertical Eye Movement Exercises

Once the break period ends, the device will beep twice for the next exercise. The device 100 is set up in a vertical orientation (FIG. 12B). The second exercise is the same as the first but is done in a vertical orientation. It trains vertical eye movements.

### EXAMPLE 3

### Oblique Eye Movement Exercises

Other exercises are illustrated in FIGS. 12D and 12E. These exercises are the same as the first exercise, but are done in one of the oblique orientations. They train oblique eye movements.

### EXAMPLE 4

### Binocular Vision Exercises

The device 100 may also be used to train eye-teaming skills or binocular vision. An observer places the nose in the recessed bridge 139 at the end of the horizontal bar 130 (FIG. 12C). This insures appropriate nose placement. After one of the exercise programs is activated, one LED is displayed at a time, creating an impression of movement. The observer's eyes focus on each displayed LED, leaving the LED as it is turned off and focusing on the next turned on LED. This exercise trains the eyes to work efficiently as a team, expanding the range of binocular vision. The exercise also trains the ability to aim, focus and track more accurately and efficiently. The eyes naturally aim, track, focus and work together simultaneously. By exercising their ability to track a moving target all these functions are trained at the same time. By adding the alternating red and blue LED's the focusing and convergence mechanisms are gently rocked to one side and then the other of a desired center point, or point of perfect balance. The use of alternating red and blue LED's trains the visual system to continually “let go” of its point of fixation and move on to the next stimulus.

The preferred device of the invention may come with a special pair of eyeglasses with interchangeable red and blue (or violet) lenses. When these eyeglasses are used in combination with the red and blue LED's used in the device, a special cancellation effect occurs. The eye behind the red lens only sees the red LED, while the eye behind the blue lens only sees the blue LED. When these red/blue glasses are worn while tracking alternating red and blue LED's in an eye exercise program, a unique cancellation effect occurs. Each eye alternately exercises its individual ability to accurately and efficiently aim, focus and track a target, while simultaneously reinforcing its ability to work together as an equal partner with the other eye.

By using red/blue glasses in combination with alternating red and blue LED's, the user is able to alternately train each eye to become the lead eye, at any given moment. This exercise establishes a high degree of balance between the eyes by equalizing the contribution of each eye while the two eyes are working together. Additionally, by interchanging the lenses, you increase the effect experienced by each eye individually and further balance the ability of both eyes to work as a team. These special red/blue glasses can be used while doing any of the eye exercises recommended. When red/blue glasses are used in combination with alternating red and blue LED's, it results in the eyes alternately being switched on and off the fixation target. This process re-establishes the eye's natural fusional reflex so that the eyes once again begin seeing instinctively, accurately and effortlessly. Since the brain naturally receives signals from each eye in an alternating fashion, this exercise reinforces the natural coordination of the eyes and their inherent alternate information processing nature.

### EXAMPLE 5

### Exercise Sequences 1-3

The sequence programs 1-3 shown below are non-limiting examples of preset sequences. In each program, one LED is activated at a time. The order of display is shown from left to right, with LEDs 120 numbered from 1 to 12:

### Sequence Program I

LEDs 120 are displayed one at a time in the sequence

1→2→3→4→5→6→7→8→9→10→11→12→11→10→9→8→7→6→5→4→3→2→1→ . . . for 1½ minutes. Depending on the selected speed, the cycle repeats one, two or more times during the 1½ minute exercise sequence.

### Sequence Program II

The LED's 120 are displayed one at a time in the sequence 1→12→2→11→3→10→4→9→5→8→6→7→5→8→4→9→3→10→2→1→12→ . . . for 1½ minutes. Depending on the selected speed, the cycle repeats one, two or more times during the 1½ minute exercise sequence.

### Sequence Program III

The LED's 120 are displayed randomly for 1½ minutes.

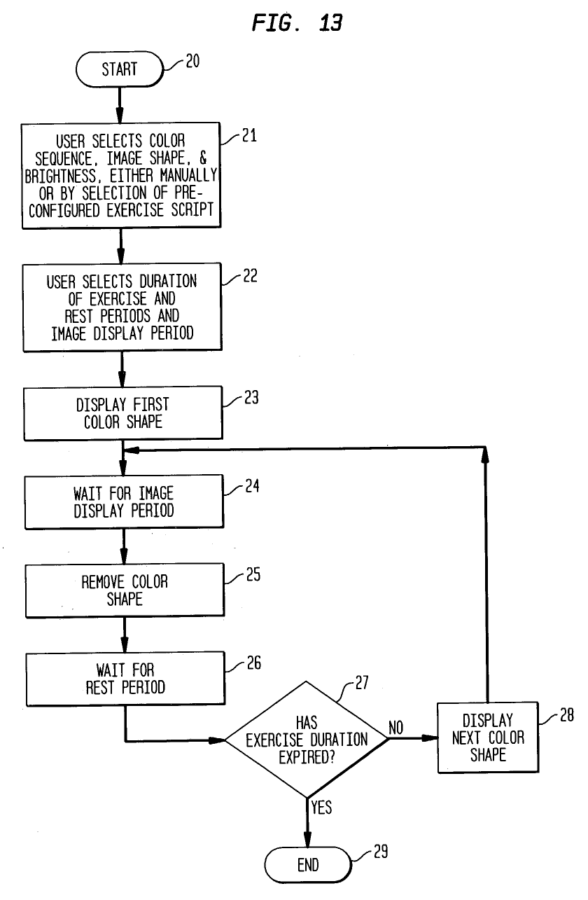

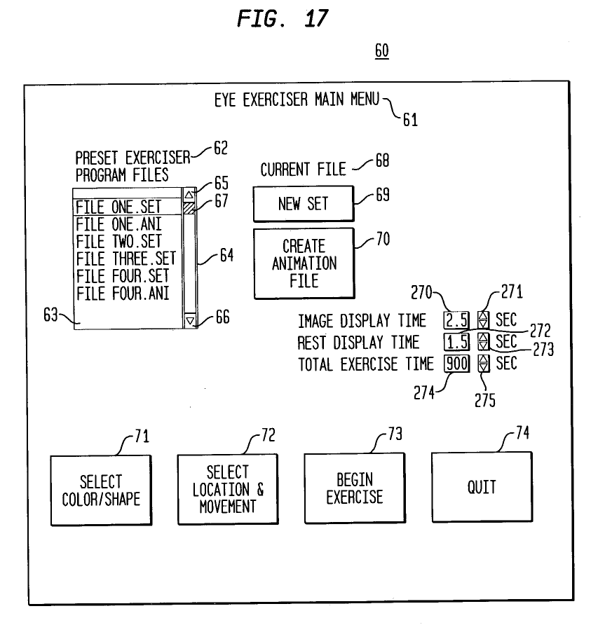

FIG. 13 is a flowchart depicting an exemplary sequence for exercising a user's eyes by changing the color of a displayed image, in accordance with a preferred embodiment of the present invention. Such images may be displayed on any display device, such as a computer display, a television monitor or similar display device, under the control of a processor executing a program incorporating the described procedure. Alternatively, the images may be displayed from a fixed program, such as a broadcast TV program, or played back from VHS tape, digital versatile disk (DVD) or via a video on demand system. In the case of a computer-controlled implementation, the user may create a custom set of color sequence, image shape and brightness using an automated setup display, such as the exemplary setup user interface (UI) screens depicted in FIGS. 17, 18 and 19, as described in detail below. The user may also preferably choose to use a pre-selected set of colors, image shape and brightness. Pre-selected sets are preferably made available to the user, and comprise sequences optimized for effectively exercising the user's eyes.

In a preferred embodiment, the user may exercise their eyes using a system including a computer connected over a communications network, such as the Internet, to a centralized site, which stores or supplies the exercise display information. The user of such a system may then log onto the system and be presented with exercise images.

In a preferred embodiment, the user selects the time periods for the exercise, for each image display, and for rest between images 22, using the setup UI screen of FIG. 17. If the user has elected a pre-selected set of colors, image shape and brightness, the pre-selected set also preferably includes default values for these time periods. In such a case the user can accept the default time periods or choose to change them.

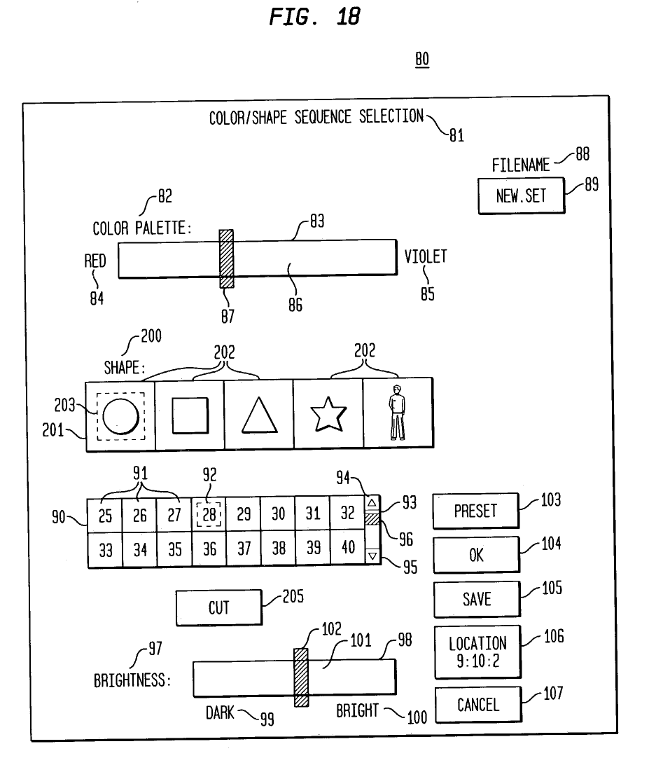

Using the exemplary setup UI screen of FIG. 18, the user may select the sequence of colors, the image shape and brightness to be displayed 21 on the display device.

The user manually entering the time periods can preferably either determine a specific amount of time for the entire exercise, or not specify this value. If no time is entered for the entire exercise, the colored images to be displayed will preferably continue cycling until the user terminates the exercise program. Otherwise, the images will be displayed until the time period for the entire exercise has expired, at which point the image display is discontinued and the exercise is ended.

Likewise, the user selects the time period for each image to be displayed and the rest period between each image display. Preferably, if the user does not select a time period for these values, a suitable default value is automatically used. A suitable default value for a preferred embodiment of the present invention for the time period for each image to be displayed is in the range 0.20 to 2.5 seconds, although any time period may be used. The default value for the rest period is preferably in the range 0.0 to 2.5 seconds, allowing for the case of no rest period between image displays, although any rest time period can be used.

Default time periods may be provided to the user. As previously mentioned, one time period specified may measure how long a single image is displayed, while another measures the rest period between images. Depending on the time periods specified, a given exercise sequence may be done different number of sequence cycles within a pre-determined exercise time (e.g., in the allotted one and one half minute, the Sequence Program I may be done one, two, three or more times depending on the selected timing periods). The table illustrates exemplary display times for an image:

Time of display for image in a sequence (seconds)

2.5
2.0
1.75
1.5
1.25
1.0
.75
.50
.25
.20
Changeable display time setting: each image is displayed on for a randomly changeable period.

Once the parameters of the eye exercise have been determined, a preferred embodiment of the invention sets the display screen to a suitable background (preferably black), and displays the first color image 23. Preferably, the appearance of a new image may be accompanied by an audible cue, such as a bell ring or other sound. This functions as an aide to remind the user to focus attention on the new image. The display device is preferably equipped with an auditory mechanism, such as a speaker, capable of providing auditory stimulus. The auditory feedback, if used, serves to reinforce the eyes' ability to accurately locate the displayed light source(s). For this purpose, a sound can be generated every time an image is first displayed. The sound goes on at the exact moment the image appears visibly. Also, another audible cue, such as a beep, may optionally be used to indicate the end of the exercise period, and for other uses. For example, a number of short beeps followed by one long beep may be used to indicate that an exercise program is about to begin, etc.

The image remains visible on the display device for the display time period 24. At the end of this time period, the image is removed from the display device 25. Preferably, the image is overwritten with the background color.

Next, in a preferred embodiment, no further visual activity occurs until the rest time period has passed 26. Since the rest time period may preferably be set to 0.0 seconds, this step may be effectively skipped.

The elapsed time of the exercise is then preferably compared with the exercise duration period 27. If the amount of time elapsed is greater than the duration period, the exercise is terminated 29. Otherwise, the next color image is displayed 28, and remains for the image display period 24. The above process is thus repeated.

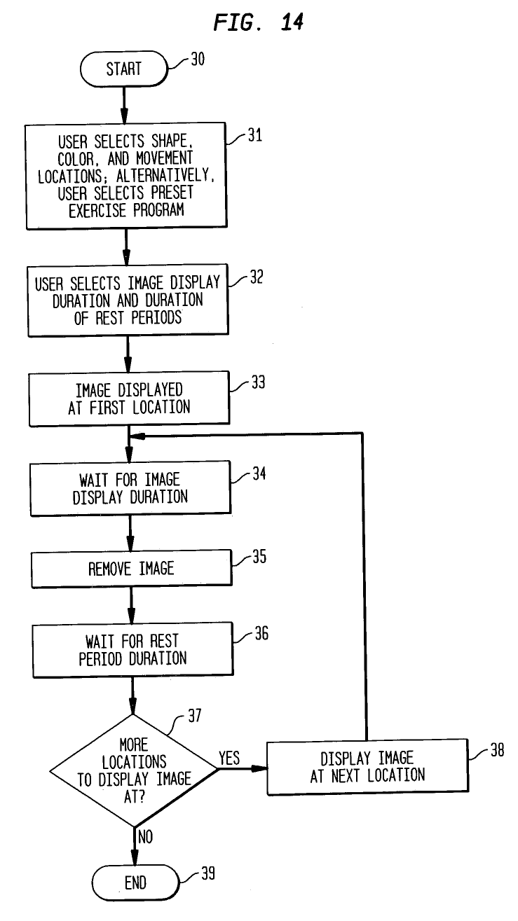

FIG. 14 is a flowchart of a similar exemplary process in a preferred embodiment of the present invention, wherein the perceived location of a displayed image is changed. At the start of the procedure 30, the user selects the shape, color and locations of the image to be displayed on the display device 31. Alternatively, as in the previous example, a preferable embodiment allows for the user to select a preset exercise program, and to select image display and rest time periods 32.

The display of the image at a perceived location is preferably enabled using three-dimensional computer rendering software, such as is commonly commercially available. Thus, three-dimensional image location and related image information is processed by such commercial rendering software to produce a resultant three-dimensional image, which is then displayed 33.

Next, after waiting for the image duration period of time 34, the image is removed 35, preferably by overwriting it with the background color.

After a delay for the rest period duration 36, if there are more locations to display the image at 37, the three-dimensional rendering software is preferably given the new image information, generates the new image for display 38. Otherwise, the exercise is terminated 39.

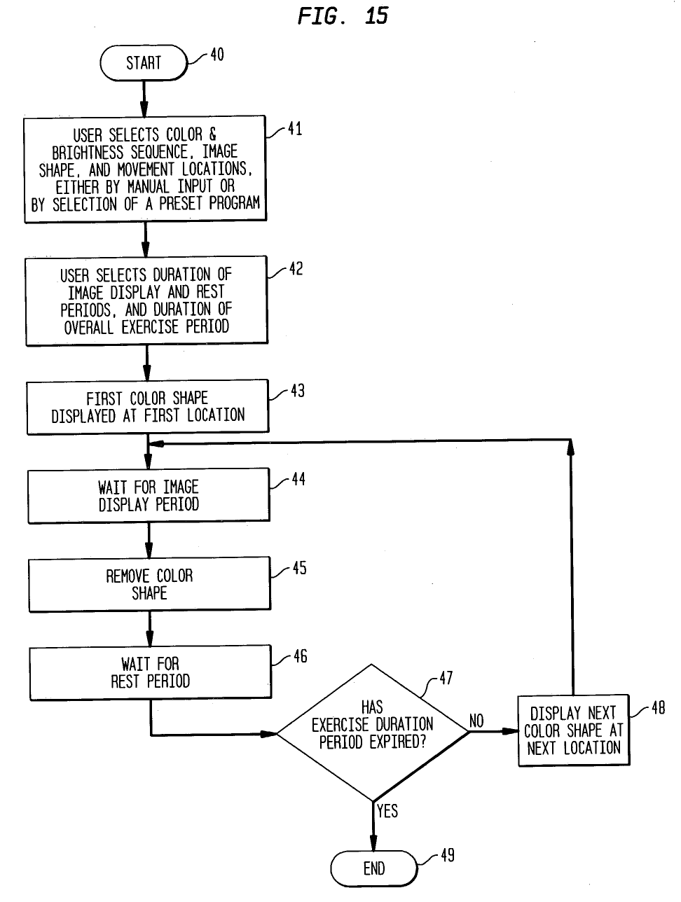

FIG. 15 is a flowchart of an exemplary process in a preferred embodiment of the present invention, wherein the both the perceived location and the color of a displayed image is changed. The procedure starts 40 when the user selects the color, brightness, shape and movement locations of the images to be displayed on the display device. The user also selects the duration of image display, the duration of rest periods, and the overall duration of exercise period 42. These selections may alternatively be made by the selection of a preset exercise program.

As in the other examples, the visual display of the image at a perceived location and in the selected color is enabled using three-dimensional computer rendering software, such as is commonly commercially available. The resulting three-dimensional image location and related image information is processed by commercial rendering software to produce a resultant three-dimensional image, which is then displayed 43.

Next, after waiting for the image display period of time 44, the image is removed 45, preferably by overwriting it with the background color.

After a delay for the rest period duration 46, if there are more images to display 37, the three-dimensional rendering software is preferably given the new image information, generates the new image for display 48. Otherwise, the exercise is terminated 49.

In a preferred embodiment, a computer may be employed to generate an animation sequence from default or user-provided color, shape, brightness, image and location information. Animation generation software is generally available employing frame interpolation methodology and other animation processes, as is known to those of ordinary skill in the art. In such processes, intermediate frames of display, generated by software, are placed between two existing, “reference” frames. For example, two reference frames of the same image, in which the image is in different locations, are used to generate one or more intermediate frames using positional interpolation. Additionally and preferably, when images change between reference frames, morphing technology may be employed to generate intermediate images. The general goal of animation techniques applied to the displays of present invention is to preferably create a smooth motion video effect for the exercise sequences, where this effect is desirable.

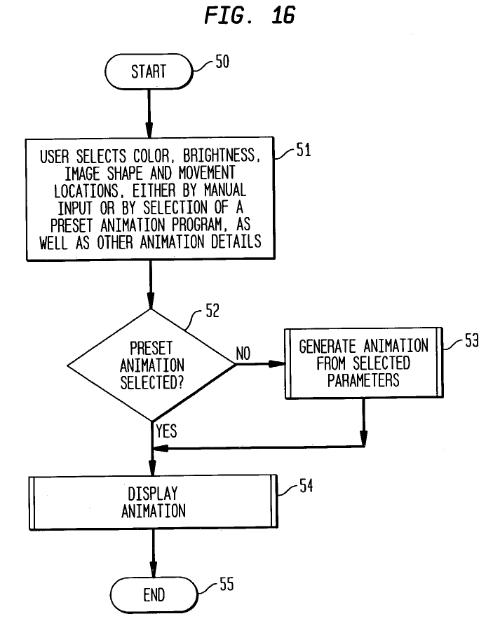

An exemplary animation and display generation sequence is shown in FIG. 16. At the start 50, the user selects the color, brightness, image shape and movement locations for a series of reference frames, or still image shots. Alternatively, default or recommended settings may be used. Additional information, such as the time between reference frames, the rest periods (if any) and the duration of the animation sequence are also provided by the user, or default values can be used. Alternatively, the user may simply select from one of a plurality of preset animation programs 51.

Next, the system determines if a preset program was selected 52. If a preset animation program was not selected, animation generation software is used to generate an animation from the user-provided input 53.

Once the animation is available, either through the immediate generation process or by retrieval of a preset animation sequence, the animation may then be displayed 54, and the process is terminated 55. The animation sequence may also be stored for future reuse. Also, the display of the animation sequence may be by any suitable display device including but not limited to a computer display screen, a television screen (preferably using a VHS tape, DVD, or other mass storage device), a head-mounted display (HMD), PDA or other electronic display devices.

FIG. 17 is a user interface (UI) screen 60 of a main menu for an exemplary software application program used to setup an eye exercise sequence. The screen 60 may be preferably implemented using standard software development tools and a general-purpose computing operating system. For example, using a Microsoft Windows operating system with Microsoft Visual C++ or Microsoft Visual BASIC can be used to create the software as described herein. Alternative operating systems and development tools may also be used, as is well known in the art of computer software development. Deviations and additions to all sample UI screens shown herein (FIGS. 17-19) due to the exact selection of operating system and development tools may occur without deviating from the scope of the invention. Moreover, additional UI screens may be used. For example, the functionality provided by the UI screen depicted in FIG. 17 may be spread over several UI screens.

An exemplary menu 60 includes a menu title 61, as well as other areas of static text 62, 68. The text displayed in these areas does not change in response to user input, and its intention is to inform the user generally of the functionality inherent in the on-screen surroundings of the text. For example, the menu title shown 61 states: “Eye Exerciser—Main Menu”. This text remains visible while the user is displaying the current screen, and it serves to inform the user what the screen is used for. Alternative fixed text can be used throughout the various UI screens presented herein without changing the functionality of the application programs. Moreover, alternative UIs may be devised and used to generate the same or similar result, and those presented herein are intended to be only exemplary in nature.

The user is presented with a scrolling list control that allows for the user to select a preset exercise program file to use. The scrolling list is a well-known type of control used in many computer applications. A general scrolling list includes an area used to display the list 63, and a scroll bar control 64 for moving the items being viewed up and down the visible area. The scroll bar control 64 preferably includes an upward button 65 and a downward button 66, which selectively move the scrolled items downward and upward, respectively. It also typically includes a positional indicator 67, which indicates the position of the visible portion of the scrollable list relative to the extent of the entire list by moving along the scroll bar area formed between the upward and downward buttons 65, 66.

A user may select an existing eye exercise program file from the scrollable list by using a mouse or other pointing device and clicking on the name of the program file to use. Double-clicking on the program file will preferably seed the program filename into the input field for the current file 69, which is positioned close to the fixed text “Current File” 68. In a preferred embodiment of the initial UI screen 60, the current file field 69 is automatically seeded with a non-preexisting filename. Other initialization options may be used in place of this. The filenames visible on the exemplary UI screens herein are examples only, and are not indicative of the allowed filenames or their contents.

The initial UI screen 60 also includes a button to generate an animation sequence 70 from an existing eye exercise program file. If an existing program file is selected in the current file field 69, selecting the animation generation button 70 will cause the existing program file to preferably be used as input to a commercial animation generator. The animation output from the animation generator may then be preferably stored as a file with a specific file extension, or type.

The main UI screen 60 also preferably includes controls for the selection of various time intervals used in a typical eye exercise sequence. For example, the user may select the period of time to display an image for, in seconds, using the image display time edit control 270, or its associated spinner control 271. The edit control, as is well known in the art, preferably works by the user first selecting the control by use of a pointing device or keyboard navigation device, then typing a value on a keyboard. Spinner controls generally function without the use of a keyboard by the user selecting either the up or down arrow with a pointer device. The selection of the up arrow of the spinner control preferably increases the value in the associated edit control, while the selection of the down arrow preferably decreases this value. Similarly, the user may preferably select the period of time between image displays using the rest display time edit control 272 and associated spinner control 273, as well as the overall period of time for the entire exercise sequence, using the total exercise time edit control 274, and its associated spinner control 275.

Other button controls are also located on the main UI screen 60. The “Select Color/Shape” button 71 will preferably invoke the “Color/Shape Sequence Selection” UI screen 80, as depicted in FIG. 18. The “Select Location & Movement” button 72 will preferably invoke the “Location/Movement Selection” UI screen 110, as depicted in FIG. 14. The “BEGIN EXERCISE” button 73 preferably initiates the selected exercise program, while the “QUIT” button 74 preferably terminates the application program. Further UI buttons and controls may be added to customize the exercise program menus as desired by the user or required by the hardware or software environment the application is used for.

FIG. 18 depicts a color shape sequence selection UI screen 80 in accordance with a preferred embodiment of the current invention. A title bar 81 appears at the upper portion of the screen 80. Selection of colors and shapes may be made on a reference frame basis. For purposes of describing the UI screens of a preferred embodiment a reference frame is encountered whenever a change in color, shape, or location is desired. A scrollable reference frame control 90 is provided which allows the selection of a particular reference frame on which to operate.

In an embodiment of the present invention, the image sequences may be created from scrollable text. The text may change colors as the user reads the image. It is believed that the action of reading the text helps to focus the user's eyes on the colored image, thus increasing the intended effect of the exercise. In this embodiment it is preferable for the text to change directly from one color to another, without blanking the display with the background color in-between.

The reference frame control 90 preferably presents the user with numbered reference frames, although alternatives to numbered frames are possible. For example, the reference frames may be presented in miniature form on the reference frame control. The reference frame control 90 of a preferred embodiment of the present invention includes a plurality of numbered frames, such as those indicated at 91. Upon user selection of a desired frame, that frame appears with an indicia of selection, such as a dashed inner frame 92. At such time, the color palette slidebar 83, shape selector 201, and brightness slidebar 98 are updated to display the currently selected color, shape, and brightness if a selection has been made. The user may also scroll through all available reference frames using the scroll bar 93 of the reference frame control 90. The scroll bar 93 includes up and down scrolling controls 94, 95, as well as a slide bar 96 indicating and controlling the displayed subset of reference controls. Scroll bars as used herein and their various possible alternative forms are well known in the art.

Once the reference frame to operate on has been selected using the reference frame control 90, the user preferably may select the image shape using the shape selector 201. Selectable shapes are depicted in boxes 202 on the shape selector 201. If too many shapes are made available to the user than can be presented by the shape selector 201, the shape selector 201 may take on an alternative form, such as a scrollable control. When the user selects a shape, an indicia of selection, such as the dashed inner box 203 of FIG. 13, is preferably used to indicate the shape selected.

Color and brightness for display of the selected reference frame are selected using the color palette slidebar 83 and brightness slidebar 98, respectively. The color palette slidebar 98 preferably represents the colors of the visible spectrum, with red 84 at one end and violet 85 at the other end of the slidebar area 86. The slidebar area 86 includes colors representing the entire visible spectrum. The granularity of the color selections available will vary depending on the ability of the display device and color graphics controller used, as is well known to those of ordinary skill in the art. The slidebar 87 itself is used to select the color by the user preferably clicking on the slidebar 87 with a pointing device and dragging it to the desired color. Alternative techniques for color selection are also possible.

Similarly, the brightness of the reference frame image selected may preferably be set using the brightness slidebar 98. The brightness slidebar preferably includes a range of grayscaling from dark 99 at one end to bright 100 at the opposite end of the brightness slidebar area 101. A pointing device may be preferably used to move the brightness slidebar 102 to the desired brightness on the brightness slidebar area 101.

A filename 87 edit control 89 is located on the color/shape sequence selection UI screen 80. This edit control 89 is seeded with the name of the file currently being worked on. If no file has been selected in a previous UI screen, this edit control 89 is seeded with a default file name. Here, the default file name “new.set” is used, although the name used is not an important aspect of the invention.

In a preferred embodiment, the user selects reference frames, colors, shapes, and brightness until no further frames or changes to frames are needed. At this point the user preferably selects the frame after the final frame needed, then selects the cut button 205. The cut button 205 truncates the available reference frames at the final frame.

At any time, the user may preferably select any of the preset 103, OK 104, save 105, location 106 or cancel 107 buttons. The cancel button 107 will cancel the color/shape sequence selection and return to the eye exerciser main menu UI screen 60. The OK button will return the user to the eye exerciser main menu UI screen 60, but maintain the color/shape selection information, so that upon return to the color/shape selection UI screen 80 the selections already made will still persist. Similarly, the save button 105 will save the current color/shape selections into the file whose filename appears in the filename edit control 89.

The preset button 103 allows the user to select the color/shapes from an existing file. For example, if the user wishes to copy an existing file's sequence changing only the shape and/or colors of the various images, the user may select the preset button 103, select the file to copy from, change the colors and/or shapes using the color/shape sequence selection UI screen 80, enter a new filename in the filename edit control 89, then save the changed sequence using the save button 105.

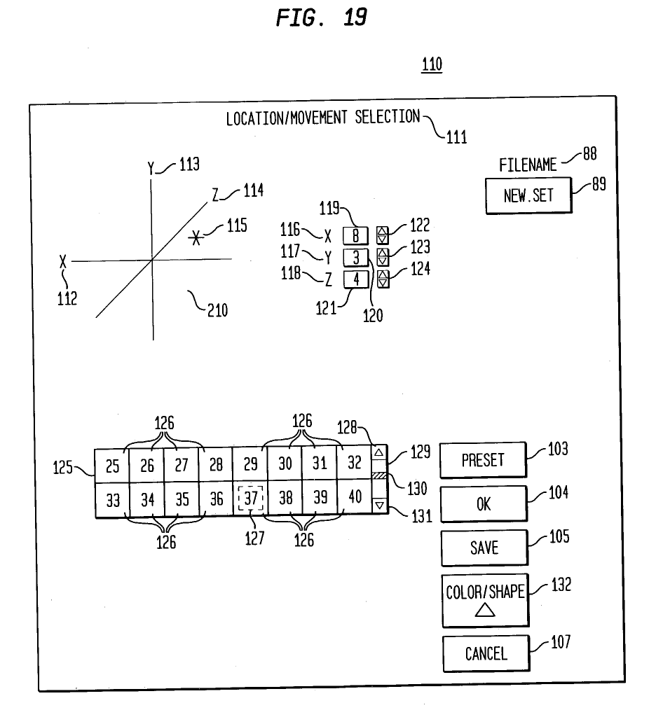

Finally, the location button 106 performs two basic tasks. When a reference image frame is selected, the location button 106 displays its three dimensional coordinates, in virtual units. The default location for a reference image is preferably centered in the foreground. Selecting the location button 106 in a preferred embodiment invokes the location/movement selection screen 110, such as depicted in FIG. 19.

The exemplary location/movement selection UI screen 110 depicted in FIG. 19 includes a title bar 111 displaying the name of the UI screen as a navigational aid to the user. It also includes a scrollable reference frame control 125 that operates in a fashion similar to the scrollable reference frame control 90 of the color/shape sequence selection UI screen 80. Thus, using the scrollable reference frame control 125, the user selects one of the available reference frames 126. The selected reference frame is preferably indicated by some indicia of selection, such as alternative shading or a dashed-line inner selection box 127. The location, in standard x, y, z coordinate format, is then preferably seeded into a three-dimensional reduced-size display 210, as well as into x, y, and z edit boxes 119, 120, 121, and spinner controls 122, 123, 124, respectively.

The three-dimensional reduced-size display 210 preferably depicts the three orthogonal coordinate axis, x, y, and z 112, 113, 114, respectively, and a marker 115 approximating the position currently selected for the chosen reference frame.

The edit boxes/spinner controls for the x, y and z coordinates 116, 117, 118 may preferably be set by the user typing the ordinal position into each of the respective edit boxes 119, 120, 121 or by use of an associated spinner control 122, 123, 124. Spinner controls are well known to those of ordinary skill in the arts of computer UI design and development.

A filename edit control 89 displays and allows the user to change the name of the file being worked on.

The user may preferably select any of the preset 103, OK 104, save 105, color/shape 132 or cancel 107 buttons at any time. The preset 103, OK 104, save 105 and cancel buttons work identically as described for the corresponding buttons in the color/shape sequence selection UI screen 80.

The color/shape button 132 performs two basic tasks. When a reference image frame is selected, the color/shape button preferably displays a miniaturized image of the shape and color of the reference frame image. Selecting the color/shape button 132 in a preferred embodiment invokes the color/shape sequence selection UI screen 80, with the currently-selected reference image frame image information selected.

By selecting the reference image frame color/shape and location/movement information, saving it, and returning to the main eye exerciser UI menu 60, the user is next ready to perform the eye exercise or generate an animation from the saved information and then perform the exercise.

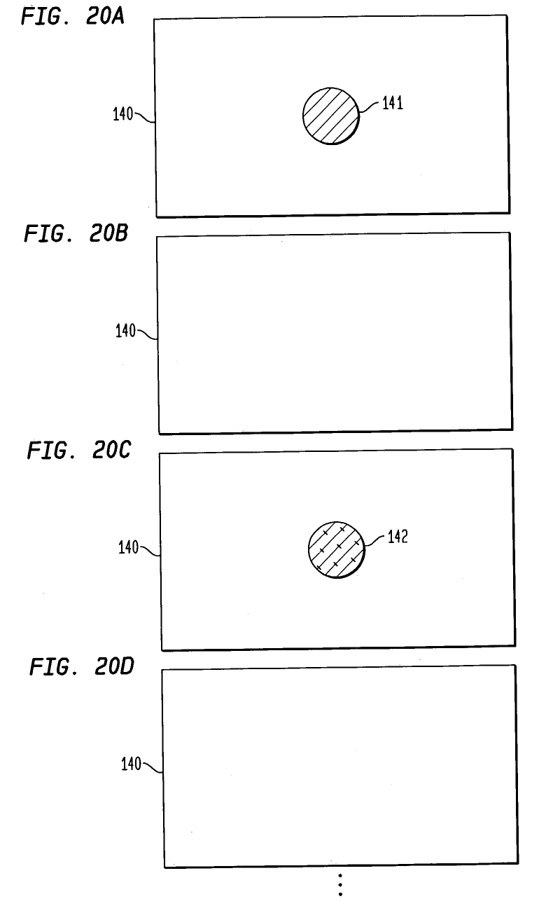

FIGS. 20(a), 20(b), 20(c), and 20(d) depict an exemplary eye exercise display color change sequence in accordance with a preferred embodiment of the invention. In this exemplary display sequence, as shown in FIG. 20(a), the display screen 140 first displays the first selected shape and color 141. After a display period, preferably set using the eye exerciser main UI screen, the display screen 140 is blanked out so as not to show the first image 141, FIG. 20(b). Once another pre-selected waiting period passes, the second selected shape and color image 142 is then displayed, FIG. 20(c). Another display period passes and this second display image is also blanked out, FIG. 20(d). This procedure continues until all the selected display images have been alternatively displayed and blanked, at which time the eye exercise is ended.

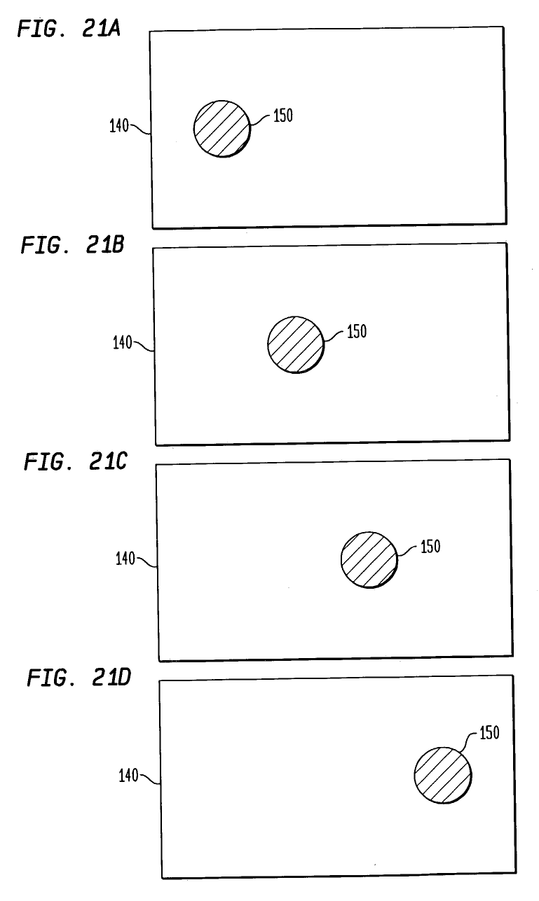

FIGS. 21(a), 21(b), 21(c), and 21(d) depict another exemplary eye exercise sequence, this one involving changes in the display image location sequence, without changing the color or shape of the image. The display image 150 is displayed at its initial location on display 140, FIG. 21(a). After a pre-selected display period, the display image 150 is erased from its initial location and re-displayed at a new location, FIG. 21(b). This process then repeats, as depicted in FIGS. 21(c) and 21(d). The sequence may then terminate, continue using new locations, or repeat for period of time determined by the user.

FIGS. 22(a), 22(b), 22(c), and 22(d) depict another exemplary eye exercise combining the changing colors of FIGS. 20(a)-20(d) and the changing locations of FIGS. 21(a)-21(d), in accordance with a preferred embodiment of the invention. The display image 160 is displayed at its initial location on display 140, FIG. 22(a). After a pre-selected display period, the display image 160 is erased from its initial location and display image 161 displayed at a new location, FIG. 21(b). This process then repeats, as depicted in FIGS. 21(c) and 21(d), displaying images 162 and 163 at their respective locations. The sequence may then terminate, continue using new colors, shapes or locations, or repeat for period of time determined by the user.

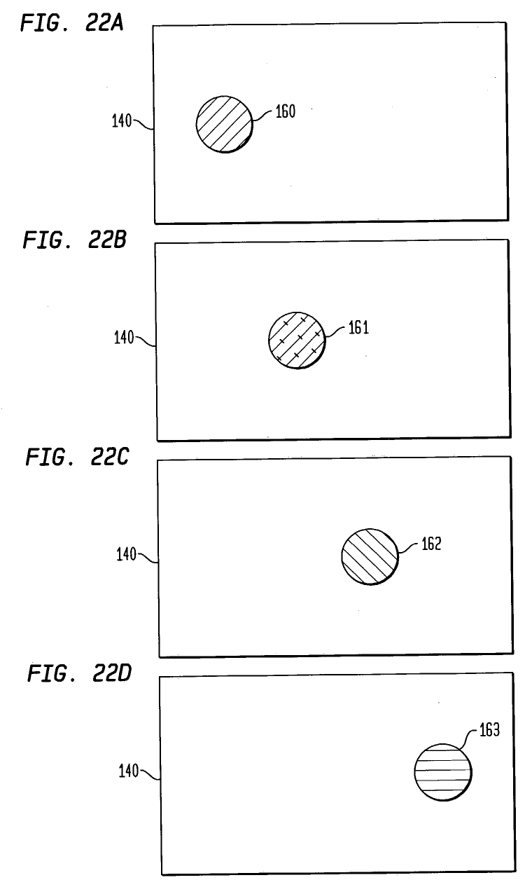

FIGS. 23(a), 23(b), 23(c), and 23(d) depict an exemplary three-dimensional eye exercise display sequence in accordance with a preferred embodiment of the invention. A line 170 has been added to the drawings to improve the three dimensional perspective. First, a first display image 171 is displayed to the user at its three-dimensional coordinates, as rendered by a commercially available three-dimensional rendering software package, FIG. 23(a). After the passage of a pre-selected display period of time, the first display image 171 is erased and a second display image 172 is displayed in its three-dimensional location, FIG. 23(b). This sequence is repeated for a third display image 173, FIG. 23(c), and a fourth display image 174, FIG. 23(d). The overall sequence may then continue for a period of time set by the user or terminate.

An exemplary eye exercise using color variation preferably includes images using at least two colors, one at a time. These include at least one image of a first color, and one image of a second color. It is preferable that the first color and the second color are selected from opposite ends of the visible spectrum. In a preferred embodiment, the first color is red, and the second color is blue or violet.

Preferably, the colored images are displayed an alternating pattern to each other. Non-limiting examples of such patterns are shown in the table:

| Color(s) | | | |
|---|---|---|---|
| of the | Color(s) of | Total | |
| first | the second | number of | |
| displayed | displayed | displayed | |
| image | image | images | Pattern* |
| R | B | 12 | R, B, R, B, R, B, R, B, R,B, R, B |
| R | V | 6 | V, R, V, R, V, R |
| R | B, V, T, I | 10 | R, V, R, I, R, B, R, T, R,V,R, B |
| R | B | 9 | B, R, B, R, B, R, B, R, B |

*R denotes red, B denotes blue, V denotes violet, T denotes turquoise, I denotes indigo.

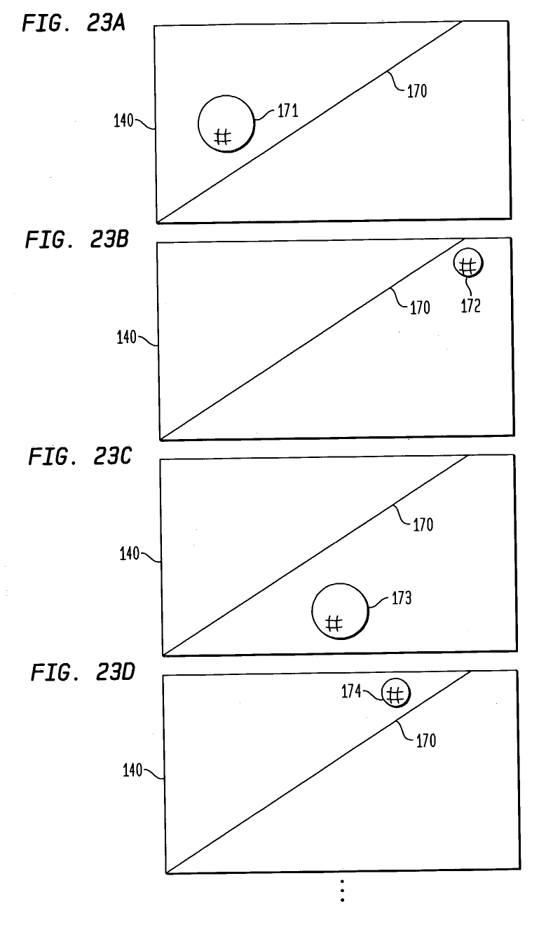

FIG. 24 illustrates a non-limiting example of the training exercise with a display of six red and six blue colored images sequentially arranged in a horizontally translated R,B,R,B,R,B,R,B,R,B,R,B pattern. O denotes the observer, and the displayed images are shown in bold. The display images are set up in a plane parallel to the observer's eyes, and displayed one-at-a-time from left to right. At the time 1, the observer perceives a red colored image at a distance al, at the time 2, a blue colored image at a distance a2, at the time 3, a red colored image at a distance a3, and so on. Thus, both the color and the distance to the target of observation (the displayed image) change during the exercise. As described, the eye adjusts its focusing power in response to both change in color and distance. The location of the displayed image in the horizontal plane relative to the observer is also changing, exercising the ability of the observer's eyes to move freely and accurately in the horizontal plane as the eyes track the movement of the displayed image.

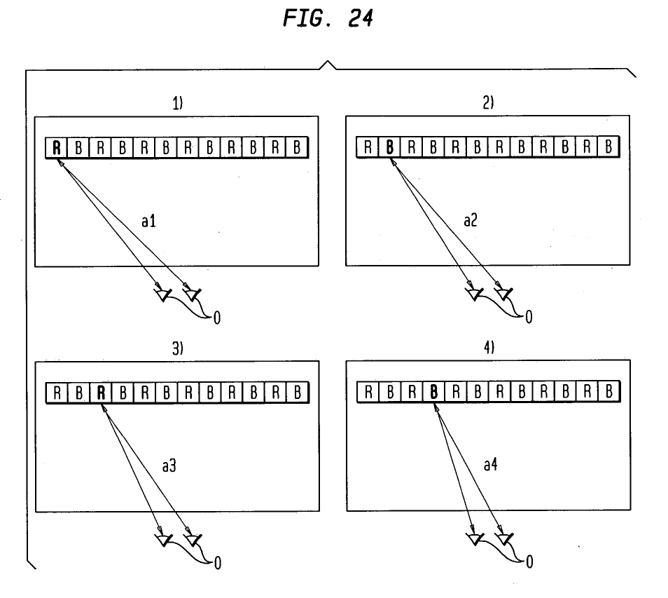

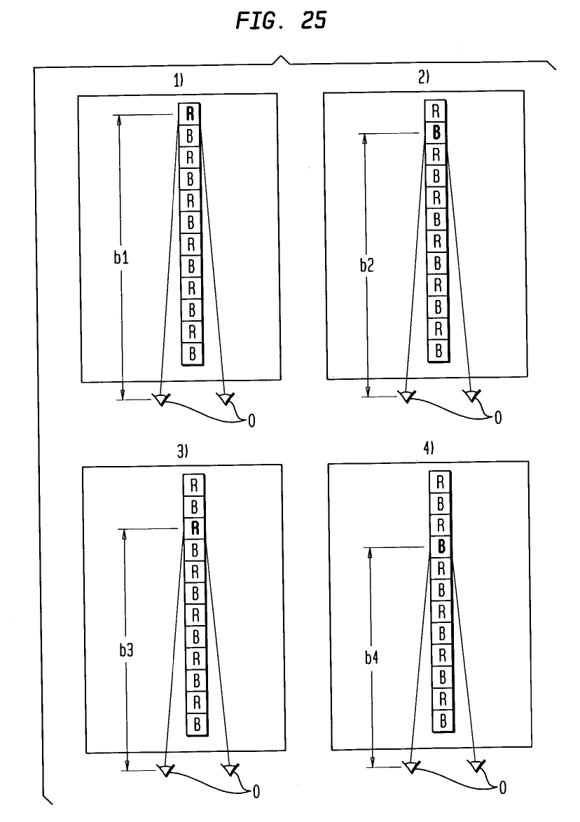

In the exercise shown in FIG. 25, the display images are placed perpendicularly to the observer. At the time 1, the observer perceives a red display image at the distance b1, at the time 2, a blue display image at the distance b2, and so on. As in the exercise shown in FIG. 24, both the color of the displayed image and the distance change. The change in the distance (e.g., from b1 to b2) is larger. In this exercise, the eyes also converge more or less as the target of observation moves closer or further, exercising the ability of the eyes to work together as a team. The use of different exercises available allows the simultaneous training of a variety of different visual skills under different conditions.

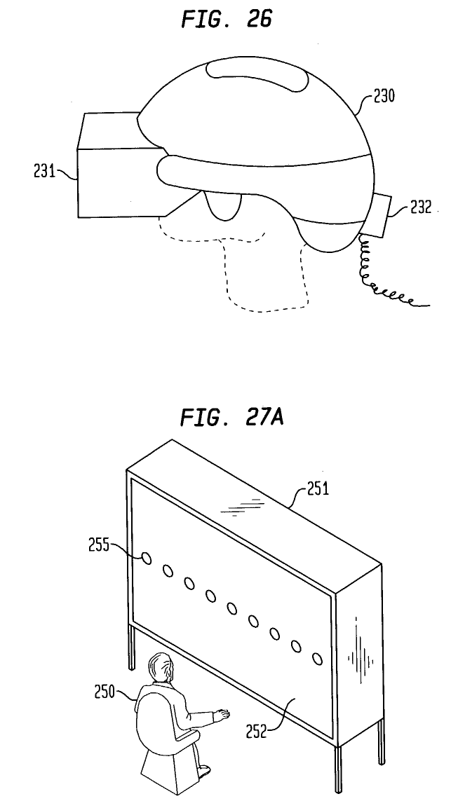

The depth perception and focusing ability of a user's eye improve as the display screen occupies a greater percentage of the user's field of view. Use of a head mounted display system, such as the one pictured in FIG. 26, is thus preferred. These systems consist of a helmet device 230 used to support a viewable display goggle 231. The helmet 230 also may have a processor or communications device 232 attached thereto, for rendering or receiving display information and controlling the display goggle 231. Head mounted display devices and their counterparts, virtual reality goggles, serve to more fully immerse the user in the perspective of a display, removing possible distractions from the exercise being performed. In the three-dimensional display of exercises involving changing image depth over time, such full immersion helps to complete the illusion of depth necessary for the optimal efficacy of the eye exercise routines. Any commercially available head mounted display or virtual reality goggles may be used in accordance with the invention. Head mounted displays are well known to those of ordinary skill in the art.

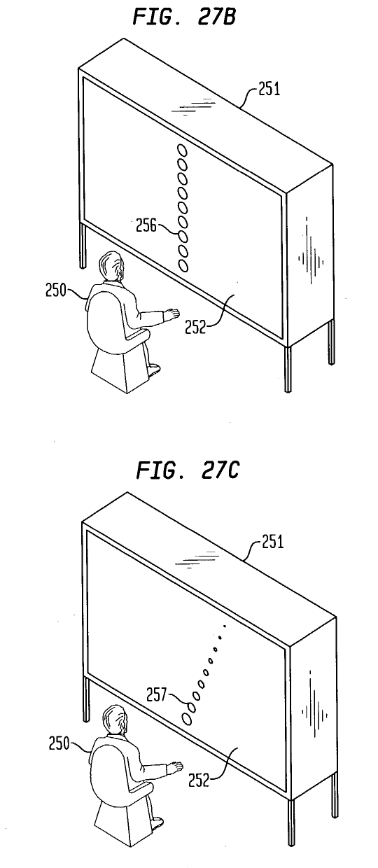

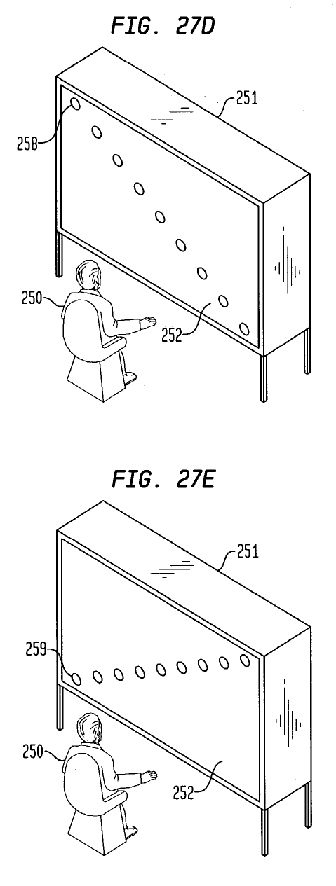

FIGS. 27(a), 27(b), 27(c), 27(d), and 27(e) depict a user 250 performing assorted eye exercises using an alternative display device 251 in accordance with a preferred embodiment of the invention. The user 250 may be seated, standing, supine or in any other position allowing an unrestricted view of the display screen 252. The display device 251 may be a large-screen television having a display screen 252, such as is commonly commercially available. The user 250 is seated in close proximity to the display screen 252, so that their field of view is largely occupied by the display screen 252. Alternatively, a smaller display device 251 having a smaller display screen 252 may be used by positioning the user 250 closer to the display screen 252, thereby preserving the proportion of the user's field of view used by the display screen 252.

In a first exemplary eye exercise, depicted in FIG. 27(a), an image 255 appears on the left side, approximately halfway down the display screen 252 and is moved to the right horizontally. This horizontal movement may be accomplished using a smooth animated translation, or by using a stepwise process as described supra. Additionally, the exercise movement may be repeated for a preset duration of time. In another exercise sequence the image 255 may first appear on the right and track leftwards horizontally.

In another exemplary eye exercise, depicted in FIG. 27(b) an image 256 appears first at the top center of the display screen 252, and then moves downwards, until reaching the bottom. Again, the movement may be viewed as a smooth animation or in a stepwise manner. Similarly, it may alternatively begin at the bottom and move upwards, and may be repeated.

FIG. 27(c) depicts an example three-dimensional eye exercise in accordance with a preferred embodiment of the invention. In this example, an image sequence 257 appears on the display screen 252, first in the foreground, with each subsequent image perceptually receding to the background. Three-dimensional rendering techniques, including those known in the art, are applied to these images to create the perception of depth. The images may singly recede to the background over time, or may similarly first appear distant and approach closer over time. The movement may be a smooth animation or may be viewed in a stepwise manner.

In two other exemplary eye exercises, depicted in FIGS. 27(d) and 27(e), the image sequences 258, 259 move diagonally across display screen 252. Again, the movement may be viewed as a smooth animation or in a stepwise manner. Similarly, it may alternatively begin at any of the corners of the display screen 252, and may be repeated.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for exercising an observer's eyes, comprising:
a display device;
a computer processor with associated computer memory, the processor operatively coupled to the display, the computer memory, and one or more input devices;
the computer processor being configured to display on the display device a predetermined sequence of colored images, the color of each colored image having varying wavelength, each successive colored image having a sufficiently different wavelength from its predecessor color image to cause the focusing power of the observer's eyes to adjust, each colored image being singly displayed for a period of time sufficient for the focusing power of an observer's eyes to adjust.

2. The system according to claim 1, wherein the computer processor is further configured to
display each colored image on the display device to the observer in a perceived location for a period of time sufficient for the observer's eyes to adjust their focusing power;
move the colored image to a different perceived location for a period of time sufficient for the observer's eyes to again adjust their focusing power; and
repeat the movement of the colored image multiple times for a given period of time sufficient to exercise the observer's eyes.

3. The system according to claim 1, wherein the color of the colored images is selected to alternate between a first color and a second color, wherein the first color has a spectral wavelength longer than the wavelength of the eye's peak spectral sensitivity in normal lighting conditions, and the second color has a spectral wavelength shorter than the wavelength of the eye's peak spectral sensitivity in normal lighting conditions.

4. The system according to claim 3, wherein the first color is red and the second color is blue or violet.

5. The system according to claim 3, wherein the first color is selected from the group consisting of orange and red, and the second color is selected from the group consisting of violet, indigo, turquoise, and blue.

6. The system according to claim 1, further comprising:
a network of one or more computers configured as a source of information for configuring the computer processor; and
a network adapter associated with the computer processor, the network adapter providing at least one communication channel connecting the computer processor to the network of computers, wherein the computer processor is configured with the information communicated from the network of computers.

7. The system according to claim 6, wherein the network of one or more computers is the Internet and the communication channel connects an Internet-based web page source of display configuration information to a program communicating with the computer processor, the program downloading the display configuration information from the web page source for use by the computer processor.

8. A medium having stored thereon instructions for directing a display device observed by an observer to display a sequence of colored images for exercising the observer's eyes, the instructions causing the display device to:
display a sequence of colored images on the display device, each image having a color varying from the color of the previous image, and each image being displayed for a period of time sufficient for the focusing power of the observer's eyes to adjust.

9. The medium according to claim 8, wherein the medium is computer-readable, the instructions are computer-implemented, and the display device is computer-controlled.

10. The medium according to claim 9, wherein the medium is internal to a computing device.

11. The medium according to claim 8, wherein the colored images are automatically displayed on the display device.

12. The medium as described in claim 9, wherein the computer-implemented instructions further include instructions for:
displaying an initial image at an initial perceived location to the observer; and
removing the initial image and displaying to the observer a sequence of images, each image replacing the prior image and having a perceived location varying from the perceived location of the prior image, with each image being displayed for a period of time sufficient for the focusing power of the observer's eyes to adjust.

13. The medium as described in claim 9, the computer-implemented steps comprising:
displaying an initial colored image at an initial perceived location to the observer; and
removing the initial colored image and displaying to the observer a sequence of colored images, each colored image a different color from the prior colored image and replacing the prior colored image and having a perceived location varying from the perceived location of the prior image, with each image being displayed for a period of time sufficient for the focusing power of the observer's eyes to adjust.

14. A computer-implemented method for exercising an observer's eyes through observation of colored images on a display device, by way of a computer-implemented program, a predetermined sequence of colored images, the color of each colored image having varying wavelength, each successive colored image having a sufficiently different wavelength from its predecessor color image to cause the focusing power of the observer's eyes to adjust, each colored image being singly displayed for a period of time sufficient for the focusing power of an observer's eyes to adjust.

15. The computer-implemented method according to claim 14, wherein the color of the colored images is selected to alternate between a first color and a second color, wherein the first color has a spectral wavelength longer than the wavelength of the eye's peak spectral sensitivity in normal lighting conditions, and the second color has a spectral wavelength shorter than the wavelength of the eye's peak spectral sensitivity in normal lighting conditions.

16. The computer-implemented method according to claim 15, wherein the first color is red and the second color is blue or violet.

17. The computer-implemented method according to claim 15, wherein the first color is selected from the group consisting of orange and red, and the second color is selected from the group consisting of violet, indigo, turquoise, and blue.

18. A method of exercising an observer's eyes, said method comprising displaying to the observer a predetermined sequence of colored objects that comprises at least a first colored object and a second colored object, wherein the color of the second colored object is different from the color of the first colored object;
the colors of the first colored object and the second colored object having spectral wavelengths on different sides of peak spectral sensitivity of a human eye in normal lighting conditions.

19. The method of claim 18, wherein the color of the first colored object has a spectral wavelength longer than the wavelength of said peak spectral sensitivity and the color of the second colored object has a spectral wavelength shorter than the wavelength of said peak spectral sensitivity.

20. The method of claim 18, wherein the color of the first object has a spectral wavelength shorter than the wavelength of said peak spectral sensitivity and the color of the second colored object has a spectral wavelength longer than the wavelength of said peak spectral sensitivity.

21. The method of claim 18, wherein the color of the first colored object is red and the color of the second colored object is blue.

22. The method of claim 18, wherein the color of the first colored object is blue and the color of the second colored object is red.

23. The method of claim 18, wherein the color of the first colored object is red and the color of the second colored object is indigo.

24. The method of claim 18, wherein the color of the first colored object is indigo and the color of the second colored object is red.

25. The method of claim 18, wherein the predetermined sequence further comprises a third object having color different than the color of the second colored object.

26. The method of claim 25, wherein the color of the third colored object is located on a different side of said peak spectral sensitivity than the color of the second colored object.

27. The method of claim 26, wherein the predetermined sequence comprises a plurality of colored objects greater than three.

28. The method of claim 27, wherein the color of each successive colored object in the pre-determined sequence is located on a different side of said peak spectral sensitivity from the color of each immediately preceding colored object.

29. The method of claim 28, wherein each colored object is displayed one at a time for a period of time sufficient for the focusing power of the observer's eyes to adjust.

30. The method of claim 18, wherein each colored object of the pre-determined sequence is an image produced on a display device for perception by the observer.

31. The method of claim 18, wherein each object of the pre-determined sequence is a physical object.

32. The method of claim 18, wherein the color of the first colored object consists of a single color and the color of the second colored object consists of a single color.

33. The method of claim 18, further comprising displaying the second colored object at a location different from a location of the first colored object, wherein the colored objects are displayed at each location for a period of time sufficient to adjust the focusing power of the observer's eyes.

34. The method of claim 33, wherein each successive colored object in the pre-determined sequence is displayed at a different location from each preceding colored object.

35. The method of claim 18, wherein the second colored object has a shape different from the first colored object.

36. The method of claim 35, wherein each successive colored object in the sequence has a shape different from each preceding colored object.
